(19) United States
(12) Patent Application Publication
(10) Pub. No.: US 2023/0223145 A1
(43) Pub. Date: Jul. 13, 2023

(54) METHODS AND SOFTWARE SYSTEMS TO OPTIMIZE AND PERSONALIZE THE FREQUENCY OF CANCER SCREENING BLOOD TESTS

(71) Applicant: 20/20 GeneSystems, Gaithersburg, MD (US)

(72) Inventors: Jonathan Cohen, Potomac, MD (US); Michael Lebowitz, Gaithersburg, MD (US); Jiming Zhou, Gaithersburg, MD (US); Hsin-Yao Wang, Chiayi City (TW)

(73) Assignee: 20/20 GeneSystems, Gaithersburg, MD (US)

(21) Appl. No.: 18/007,725

(22) PCT Filed: Jun. 1, 2021
(86) PCT No.: PCT/US21/35251

§ 371 Date: Dec. 1, 2022

Related U.S. Application Data
(60) Provisional application No. 63/033,192, filed on Jun. 1, 2020.

(51) Int. Cl. G16H 50/20; G16B 25/10; G16H 50/30; G16H 10/40

(52) U.S. Cl. CPC G16H 50/20; G16B 25/10; G16H 50/30; G16H 10/40

(57) ABSTRACT

Disclosed herein are classifier models, computer implemented systems, machine learning systems and methods thereof for classifying asymptomatic patients into a risk category for having or developing cancer and/or classifying a patient with an increased risk of having or developing cancer into an organ system-based malignancy class membership and/or into a specific cancer class membership and/or a category with a time range for follow up testing or reclassification with newly measured input factors.

### RELATED APPLICATIONS

This application claims priority to provisional application U.S. Ser. No. 63/033,192 filed 1 Jun. 2020, which is hereby incorporated into this application in its entirety.

### FIELD OF THE DISCLOSURE

This application pertains generally to classifier models generated by a machine learning system, trained with longitudinal data, for identifying asymptomatic patients with an increased risk for developing cancer, by recommending repeat or serial retesting at varying time intervals.

### BACKGROUND OF THE DISCLOSURE

Early detection of cancer is one of the major keys to improving survival of patients by enabling early treatment, including surgical removal of the localized solid tumor, before metastasis when survival rates reduce sharply to less than 50% even with state-of-the-art systemic therapies [1]. Many cancers can take years to develop to metastasis from their original lesions [2], providing the opportunity for detection of cancer at this early stage. Several tools are currently used for cancer screening [3], such as low-dose chest computed tomography (CT) for lung cancer, mammography for breast cancer, pap smear for cervical cancer, and stool occult blood for colorectal cancer. The performance of these tools is variable. This is in part due to their operator-dependent nature [4,5], limited availability, and difficulty of use. For example, pronounced disparities in the availability of low-dose chest CT instrumentation restrict its wide application [6], while the collection of specimens by non-medically trained individuals jeopardizes the accuracy of stool occult blood testing [7]. Furthermore, these tools usually detect only one cancer type, meaning that individuals may need to visit multiple medical services to receive different screening tests. These disadvantages lead to low compliance with cancer screening by these tools [8]. Additionally, these screening approaches are impractical for testing more than once every year or two even though some tumors might metastasize at a rate that could benefit from retesting at shorter time intervals.

Serum protein tumor markers like CEA, AFP, CA-125, CA-19.9, PSA, etc., have been used for decades to aid in the diagnosis and management of a variety of cancers. Except for PSA, most international guidelines recommend routine use of these markers for monitoring cancer recurrence or therapy response, but not for screening or early detection [3]. Nevertheless, in parts of Asia, these tumor markers are routinely measured as part of yearly physical exams for tens of millions of individuals each year and have been successfully used for the early detection of cancer [3,9]. According to feedback from “health check-up” and physical examination centers in Japan, Taiwan, Korea, China, and Russia, the popularity of this testing approach appears to be growing.

Over the past 30 years tens of billions of dollars have been invested to discover and validate alternatives to serum protein tumor markers. The alternative targets include circulating tumor DNA, microRNA, and circulating tumor cells [10-12]; yet to date, none of these approaches has seen widespread clinical adoption, either because of cost or a lack of prospective or real-world validation. In order to accurately assess the efficacy of the tests in a real world asymptomatic population, it is absolutely crucial to use real world evidence (RWE) derived from real world data (RWD) to validate findings from case-controlled studies and to generalize the findings back to real world situations [13-15]. As immunological measurement of tumor markers has been performed over a number of years on a large population of individuals in a pre-diagnostic mode, RWD now exists for these biomarkers. While single tumor markers may not perform well enough, using a marker panel consisting of multiple tumor markers can significantly improve the performance of cancer screening tests [3,9-10]. Thus, tumor marker measurement is now routinely performed in Eastern Asia and has resulted in the early detection of cancers in the asymptomatic population.

Supervised machine learning (ML) is a good analytical method for solving classification problems through identification of implicit data patterns from complex data. The ML method outperforms some traditional statistical methods (i.e., univariate analysis) because of its excellent ability to handle complex interactions between large numbers of predictors and good performance in non-linear classification problems. ML has been successfully applied in several clinical fields and outperforms traditional statistical methods [16-18].

It is known and understood with protein tumor markers that readings at a single time point are of limited value. Rather changes in marker values following successive tests enhances sensitivity and specificity. Unfortunately, unless tumor marker levels are abnormally high, or the patient displays signs or symptoms of a malignancy, testing is repeated at one- or two-year intervals.

It would be desirable to have scientifically valid methods for determining who might benefit from repeat or serial tumor marker testing at intervals shorter than one year so as to increase the likelihood of detecting tumors at an earlier stage thereby improving outcomes. Such methods are provided by this disclosure.

### SUMMARY OF THE DISCLOSURE

Disclosed herein are methods and software systems to optimize and personalize the frequency of cancer screening blood tests including, without limitation, classifier models, machine learning systems, computer implemented systems and methods thereof. Provided are a first classifier model, a second classifier model and a third classifier model. The first classifier model may be used to classify (asymptomatic) patients into a risk (e.g., high, elevated, etc.) or non-risk category for having or developing cancer. In embodiments, the second classifier model may be used to identify a likely organ system of the cancer. In certain other embodiments, provided herein is a third classifier model, which comprises the first classifier model, and may be used to categorize subjects into a category including time ranges for follow-up testing or re-classification using the first classifier model with updated inputs (e.g. newly measured biomarkers and updated age). In short, disclosed herein is a method for screening for cancers in an asymptomatic human subject comprising:
a. obtaining a first blood sample from the human subject;
b. measuring a panel of at least two markers in the sample, wherein said markers are selected from the group consisting of CEA, AFP, CA125, CA15-3, CA19-9, Cyfra, and PSA;
c. providing machine learning software to produce a cancer likelihood score, wherein said software is built from data from individuals previously tested with said marker panel and for which cancer outcomes are known;
d. generating a cancer likelihood score for the human subject;
e. using said cancer likelihood score to calculate the optimal time interval when said at least two markers should be re-measured in a second blood sample from the human subject;
f. obtaining a second blood sample from the human subject based on said time interval; and
g. re-measuring said panel of markers in the second blood sample and comparing the changes in marker levels between the first and second blood samples.

In certain embodiments, this disclosure provides methods for identifying a patient for follow-up cancer diagnostic testing, the method comprising: a) assigning a risk score of having or developing cancer to the patient, wherein the risk score is generated using a first classifier model using input variables of measured values of a panel of biomarkers from the patient and clinical factors including at least age and a diagnostic indicator, for a population of patients, when an output of the first classifier model is a numerical expression of the percent likelihood of having or developing cancer; b) classifying the patient into an increased risk category of having or developing cancer when their risk score, generated by the first classifier model, is above a first pre-determined threshold, wherein the first pre-determined threshold is a prevalence of cancer in the population of patients; c) classifying those patients in the increased risk category into a follow-up category using the risk score generated by the first classifier model, wherein a second pre-determined threshold, and optionally third pre-determined threshold, separate the follow-up categories and the second, and optionally third, pre-determined threshold is a median time to definitive diagnosis following measurement of the biomarkers of the population of patients; and, d) providing a notification to a user of the patient risk score and follow-up category, wherein follow-up testing is selected from repeat testing in about one year, repeat testing in less than about 1 year and/or confirmatory cancer diagnostic testing.

In certain embodiments provided herein is a computer-implemented method for generating a follow-up cancer diagnostic testing classifier model comprising: a) obtaining, by one or more processors, a data set from a population of patients comprising a risk score of having or developing cancer and time to definitive diagnosis following measurement of one or more biomarkers, wherein the risk score is generated by a first classifier model using inputs of measured values of the one or more biomarkers, optionally age, and a diagnostic indicator, from a population of patients; b) segmenting the data into two or more groups based on the risk score; and, c) determining a median time to definitive diagnosis in each group; and, d) generating the classifier model for follow-up cancer diagnostic testing based on the correlation between the risk score and the time to definitive diagnosis, wherein the classifier model provides output selected from repeat testing in about one year, repeat testing in less than about 1 year and/or confirmatory cancer diagnostic testing.

### BRIEF DESCRIPTION OF THE DRAWINGS

The drawings illustrate generally, by way of example, but not by way of limitation, various embodiments disclosed herein.

FIG. 1 shows: (a) Receiver operating characteristic (ROC) curves of internal cross validation of the cancers screening algorithms by male training dataset. LR: logistic regression; RF: random forest; SVM: support vector machine; (b) ROC curves of external validation of the cancers screening algorithms by male independent dataset; (c) ROC curves of cancers screening algorithm for male different cancer types; (d) ROC curves of internal cross validation of the cancers screening algorithms by female training dataset; (e) ROC curves of external validation of the cancers screening algorithms by female independent dataset; and, (f) ROC curves of cancers screening algorithm for female different cancer types.

FIG. 2 shows use of an organ system algorithm for localizing tissue origin. For the cases predicted with non-low risk scores, a k-nearest neighbor based organ system algorithm was used for localizing tissue origin. The performance of a different number of origins is evaluated. When only one possible tissue origin (i.e., top 1) is reported, the sensitivity is low, but specificity is high. In contrast, the sensitivity increases, and specificity decreases when more possible tissue origins (e.g., top 10) are reported. A balanced sensitivity and specificity of the algorithm can be achieved when the top three possible organ systems are reported.

FIG. 3 shows: (a) Characteristics for different levels of risk wherein the distribution of stages of cancers for different levels of risk (male) is illustrated; and, (b) Characteristics for different levels of risk. The distribution of stages of cancers for different levels of risk (female) is illustrated. The risk score for both the male and female groups is positively correlated to the cancer stage, namely the proportion of advanced cancer stage increases in the higher risk score categories.

FIG. 4A shows Time to Diagnosis (TTD) for cancer cases with different levels of risk (male). The median TTD is inversely correlated to the risk score: when a case is predicted with a higher risk score, a shorter TTD can be expected, thus a shorter time for additional investigation could be suggested.

FIG. 4B shows Time to Diagnosis (TTD) for the cancer cases with different levels of risk (female). The median TTD is inversely correlated to the risk score: when a case is predicted with a higher risk score, a shorter TTD can be expected, thus a shorter time for additional investigation could be suggested.

FIG. 5 shows actionable recommendation for the different levels of risk scores. When low risk is reported and all the biomarkers are below the reference ranges, 1-year follow-up is recommended. For the low risk group whose marker is elevated and for the mild risk group, 1-month repeat is recommended to exclude an elevated risk caused by interference. For the individuals whose risk score keeps elevating upon repeat and for the moderate risk group, 6-month follow-up in the suggested specialties (i.e., top 3 most likely affected organ systems provided by the Organ System Algorithm) is recommended. For the high risk group, 2-month or shorter follow-up is recommended for further investigation of cancer.

FIG. 6 shows data preparation with male as an example, and female has the same structure. For each subsampling, the cancer cases were first split into training and validation datasets by ratio 70:30; for training dataset, the same number of non cancer cases were randomly taken from the whole non cancer cases in Linkou branch, and the rest of the non cancer cases were partitioned to the validation data set. Thus, for male model, the training data had 87 cancer cases and 87 non cancer cases, the validation data had 37 cancer cases and 8204 non cancer cases, whose cancer versus non cancer case ratio remained as the same as the original dataset from Linkou branch. After the training and internal cross validation for 200 times, all the 124 cancer cases and 124 non cancer cases were randomly selected to build the cancer screening ML models. The data collected from Kaohsiung branch were used as the independent testing dataset to test the robustness of the ML models.

FIG. 7 shows cutoff or threshold values for males (LR algorithm) and females (RF algorithm).

FIG. 8. Shows Time to Diagnosis (TTD) for cancer cases with different levels of risk (Elevated (wherein “elevated” correlates to combined low, mild and moderate groups in model of FIG. 7) and High risk groups). The median TTD is inversely correlated to the risk score: when a case is predicted with a higher risk score, a shorter TTD can be expected, thus a shorter time for additional investigation could be suggested.

### DETAILED DESCRIPTION OF THE DISCLOSURE

### Introduction

Provided herein are computer-implemented methods for generating a follow-up cancer screening classifier model and methods for identifying a patient for follow-up cancer screening.

Using the largest reported database of real word data and external validation, we developed and validated ML-derived software that substantially improves tumor biomarker testing by incorporating the values of two or more biomarkers with age and gender to assign level of risk for cancer. Further, a secondary model was developed to predict the top three most likely affected organ systems for individuals identified as at-risk. The ML algorithms are robust and help detect cancers at the earliest stages in asymptomatic individuals.

Immunoassays for tumor markers have been developed over the last several decades, and most are used for monitoring during post-therapy follow-ups but not in a pre-diagnostic mode for screening of asymptomatic individuals. Using single tumor biomarkers for cancer screening has been less robust; and even PSA, the only tumor marker widely used for cancer screening, remains controversial [25]. Clinicians generally interpret lab values by a “single threshold method”, which is based on pre-determined reference ranges for each individual marker. However, the reference ranges are set solely based on the value distribution within the normal population, not for cancer screening, they are not adjusted in connection with other marker levels, and do not consider age, gender or any other patient characteristic. By contrast, ML algorithms can learn and identify the specific pattern of tumor markers and clinical factors and their interdependence for discriminating cancer cases from non-cancer cases.

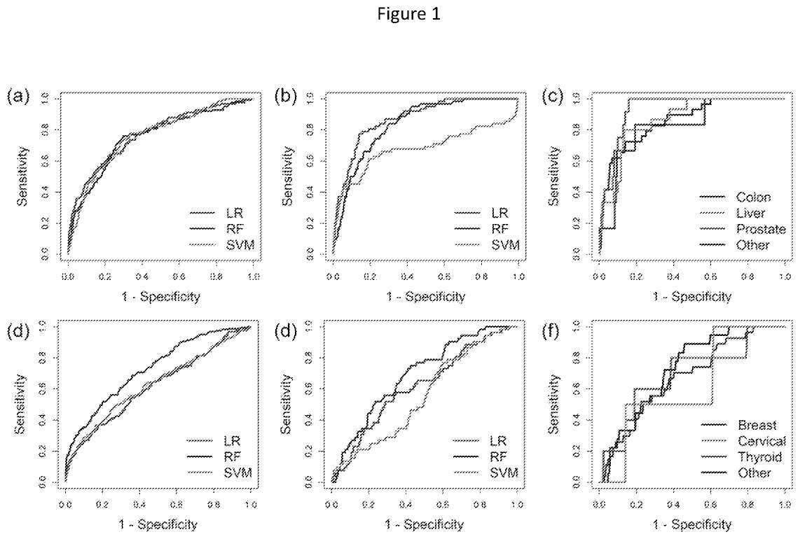

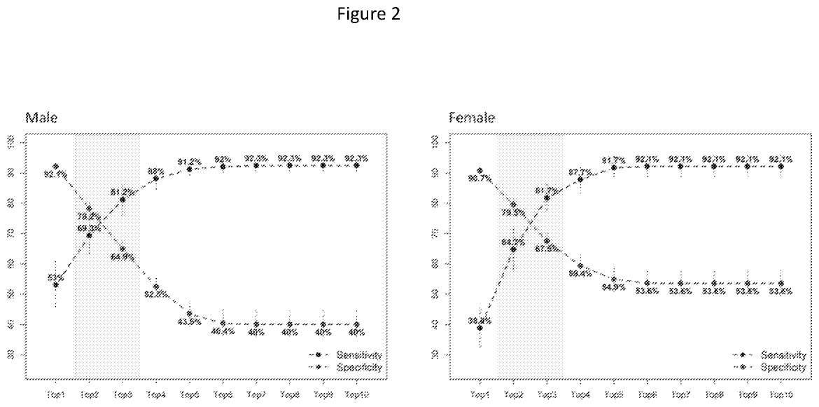

The method according to the present invention utilizes a large dataset of real world data currently available, collected over 14 years for both training and external validation of ML models to interpret tumor marker panels. These algorithms demonstrated superior performance characteristics (Table 2, FIGS. 1 and 2), and the robustness of these models was validated by external validation. In addition, all the subjects were individuals undergoing a yearly health check-up with no prior indication of cancer and thus represent a real-world asymptomatic population [3,9]. Due to the use of RWD, the ML model developed herein is ready for immediate application in the real-world setting.

In certain embodiments provided herein is a classifier model that identifies the top three most possible (more than one) organ systems for physicians or caregivers to further investigate the possible origin of cancer for a patient. Based on the design, a subject/patient will be labeled with the top three most possible organ systems, for example: “Chest”, “Ear, Nose, and Throat”, and “Gastrointestinal”, such as in the report. An experienced physician would further check the individual's specific exposure history (e.g., smoking, fine particulate matter (PM2.5)) and arrange a low-dose chest CT because the reported pattern may imply a clinical picture of cancerous change over the pharynx/larynx and lung, which can be classified to organ system labels of “Ear, Nose, and Throat” and “Chest”, respectively. Regarding the aspect of analysis and reporting, we used all the cancer cases to cross-validate the KNN model and evaluate by what N we could help physicians identify the tissue origin of cancer accurately. In embodiments, reporting only the top three most possible organ systems was based on (1) achieving the best balance between sensitivity and specificity that could be achieved (e.g., performance), and (2) a top three possible tissue origin would be reasonable and actionable for further clinical survey in most of clinical settings.

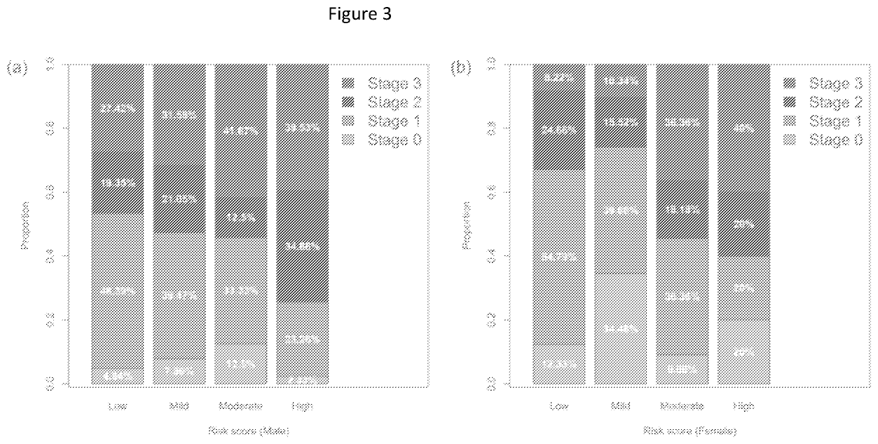

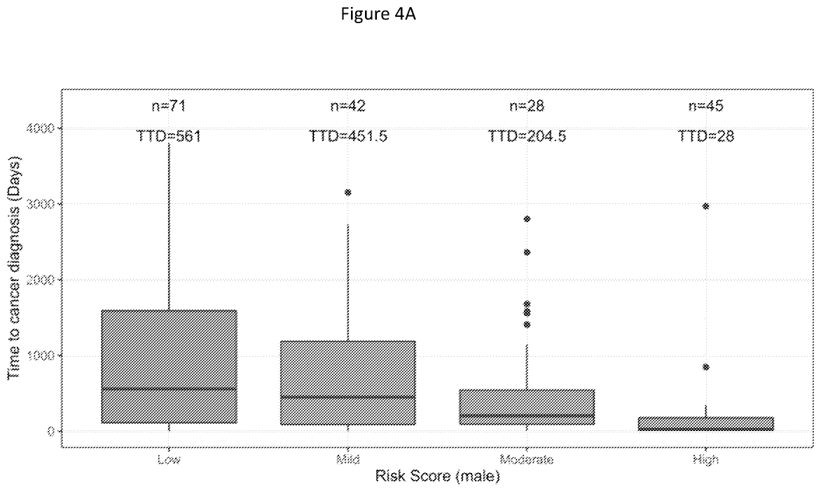

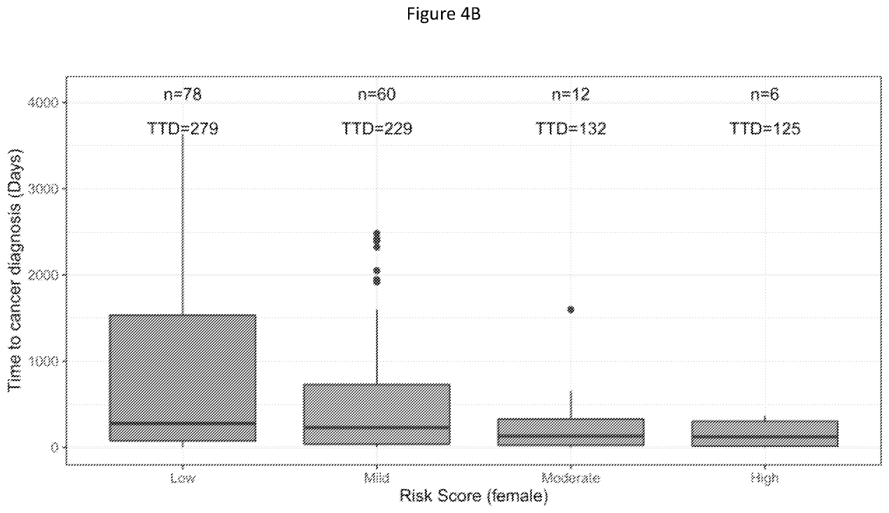

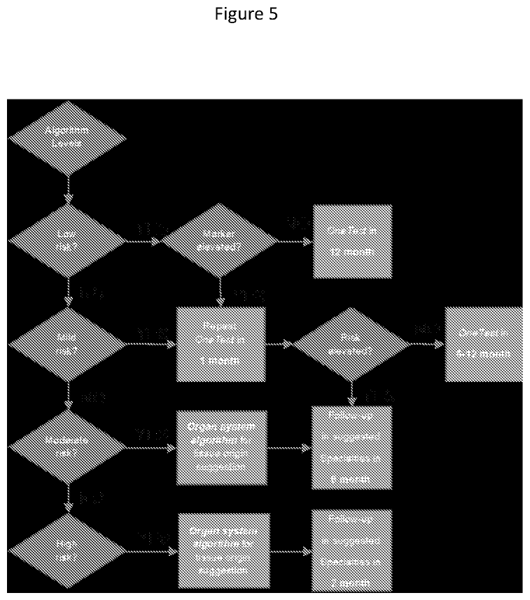

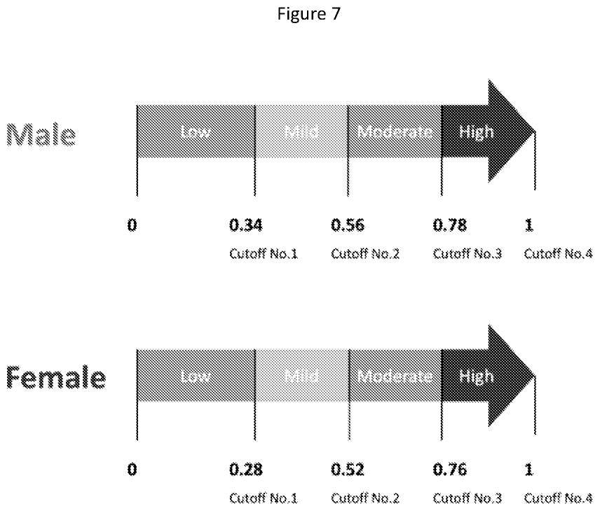

To generate a ML model usable in the clinical setting, provided herein is a two-layer model for cancer screening. The first layer ML model identifies the relative risk of individuals for developing cancer in the near term, and the second layer ML model predicts the top three most likely affected organ systems. In certain embodiments, in the first layer, four risk levels are reported. The PPVs for the different risk levels, together with their correlations to cancer stage at diagnosis (FIG. 3) and time to diagnosis (FIG. 4) suggest different follow-up procedures (FIG. 5). TTD is an important factor in determining post screening patient follow-up. The short TTD (2-4 months) for individuals in the high-risk group suggests these patients should be followed up in the short term. For individuals at both moderate and high risk, based on TTD, next steps likely should include referral to a specialist for further work-up, ideally within 6 and 2 months, respectively. Advanced diagnostic tools could be used for confirmation; for example, low-dose chest CT could be used when “chest” is predicted as the most likely organ system. The action following a positive risk call should be at the discretion of physicians and depend heavily on standard health practices in different countries. In Taiwan, a colonoscopy will be used as the diagnostic tool for following up at-risk individuals because it is affordable and available in nearly every hospital. At the same time, since many cases of mild risk may be at a pre-cancer status which is a dynamic stage between developing into a local tumor and being eliminated by the immune system, and the half-life of most tumor markers is less than several days [29], the methods of this disclosure would result in a recommendation that these individuals receive repeat tumor marker screenings in one month to verify the level of risk. Future repeat testing at 6-12-month intervals to monitor changes in overall score and individual biomarker levels would seem prudent. See FIG. 7.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term “asymptomatic” refers to a patient or human subject that has not previously been diagnosed with the same cancer that their risk of having is now being quantified and categorized. For example, human subjects may show signs such as coughing, fatigue, pain, etc., but have not been previously diagnosed with lung cancer but are now undergoing screening to categorize their increased risk for the presence of cancer and for the present methods are still considered “asymptomatic”.

As used herein, the term “AUC” refers to the Area Under the Curve, for example, of a ROC Curve. That value can assess the merit or performance of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for a ROC curve, such as, JMP™ or Analyse-It™. AUC can be used to compare the accuracy of the classification model across the complete data range. Classification models with greater AUC have, by definition, a greater capacity to classify unknowns correctly between the two groups of interest (disease and no disease).

As used herein, the term “blood sample” refers to blood or components thereof such as whole blood, serum, plasma, or a suitable fraction thereof, whether collected from a capillary, vein or artery.

As used herein, the terms “cancer” and “cancerous” refer to or describe the physiological condition in mammals that is typically characterized by unregulated cell growth. Examples of cancer include but are not limited to, lung cancer, breast cancer, colon cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

As used herein “cancer likelihood score” means a number or other quantitative measurement that indicates the tested individual's probability, risk, or likelihood of being correctly diagnosed with a cancer, or developing cancer, within a particular time period (e.g., about one year).

As used herein the “cancer outcomes” of the people whose data is used to build or validate the machine learning software includes whether there is a confirmed diagnosis of cancer using criteria generally accepted by the medical community.

As used herein “machine learning” refers to algorithms that give a computer the ability to learn without being explicitly programmed including algorithms that learn from and make predictions about data. Machine learning algorithms include, but are not limited to, decision tree learning, artificial neural networks (ANN) (also referred to herein as a “neural net”), deep learning neural network, support vector machines, rule base machine learning, random forest, logistic regression, pattern recognition algorithms, etc. For the purposes of clarity, algorithms such as linear regression or logistic regression can be used as part of a machine learning process. However, it is understood that using linear regression or another algorithm as part of a machine learning process is distinct from performing a statistical analysis such as regression with a spreadsheet program such as Excel. The machine learning process has the ability to continually learn and adjust the classifier model as new data becomes available and does not rely on explicit or rules-based programming. Statistical modeling relies on finding relationships between variables (e.g., mathematical equations) to predict an outcome.

As used herein, the term “medical history” refers to any type of medical information associated with a patient. In some embodiments, the medical history is stored in an electronic medical records database. Medical history may include clinical data (e.g., imaging modalities, blood work, biomarkers, cancerous samples and control samples, labs, etc.), clinical notes, symptoms, severity of symptoms, number of years smoking, family history of a disease, history of illness, treatment and outcomes, an ICD code indicating a particular diagnosis, history of other diseases, radiology reports, imaging studies, reports, medical histories, genetic risk factors identified from genetic testing, genetic mutations, etc.

As used herein, the term “increased risk” refers to an increase in the risk level, for a human subject after analysis by the classifier model, for the presence, or development, of a cancer relative to a population's known prevalence of a particular cancer before testing. In other words, a human subject's risk for cancer before biomarker testing and/or data analysis may be 1% (based on the understood prevalence of cancer in the population), but after analysis using the classifier model the patient's risk for the presence of cancer may be 8% or alternatively reported as an increase of 8 times compared to the cohort. The machine learning system calculates the 8% risk of having the cancer and the increased risk of 8 times relative to the population or cohort population is provided in more detail herein.

As used herein, the term “a positive predictive score,” “a positive predictive value,” or “PPV” refers to the likelihood that a score within a certain range on a biomarker test is a true positive result. It is defined as the number of true positive results divided by the number of total positive results. True positive results can be calculated by multiplying the test sensitivity times the prevalence of disease in the test population. False positives can be calculated by multiplying (1 minus the specificity) times (1—the prevalence of disease in the test population). Total positive results equal True Positives plus False Positives.

As used herein the term, “Receiver Operating Characteristic Curve,” or, “ROC curve,” is a plot of the performance of a particular feature for distinguishing two populations, patients with cancer, and controls, e.g., those without cancer. Data across the entire population (namely, the patients and controls) are sorted in ascending order based on the value of a single feature. Then, for each value for that feature, the true positive and false positive rates for the data are determined. The true positive rate is determined by counting the number of cases above the value for that feature under consideration and then dividing by the total number of patients. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied, weighted, etc.) to provide a single combined value which can be plotted in a ROC curve. The ROC curve is a plot of the true positive rate (sensitivity) of a test against the false positive rate (1-specificity) of the test. ROC curves provide another means to quickly screen a data set. As used herein, performance of the present classifier models is determined using computed ROC curves with sensitivity and specificity values. The performance is used to compare models, and also importantly, to compare models with different variables to select a classifier model with the highest accuracy as to predicting having or developing cancer, for a patient.

Classifier Models Generated by Machine Learning Systems and their Use

Disclosed herein are classifier models, generation of those models, computer implemented systems, machine learning systems and methods thereof for classifying (asymptomatic) patients; a) into a risk or non-risk category for having or developing cancer (a first classifier model); b) identification of likely cancer systems (for those classified in a risk category) (a second classifier model); and, c) follow-up guidance as to time for re-testing (a third classifier model). The machine learning system disclosed herein generated the present classifier models using longitudinal data from a cohort of over 12,000 asymptomatic male patients and over 15,000 asymptomatic female patients. See Example 1 and 2. In this instance biomarkers were measured, and follow-up of the patients was performed to provide a diagnostic indicator in the future (e.g. no cancer development, or diagnosis of a specific cancer and the length of time from testing to diagnosis (Time-to-Diagnosis (TTD)). Using biomarkers obtained months, or even years, before cancer was detected provided a powerful tool to train the classifier models resulting in highly accurate classifier models as measured by ROC curve analysis. In embodiments, training data comprises data from a group of patients with no cancer diagnosis three or more months after providing a sample. In embodiments, training data comprises data from a group of patients with a cancer diagnosis three or more months after providing a sample.

In embodiments, the cohort of asymptomatic female patients was used to train a classifier model to be used with female patients and the cohort of asymptomatic male patients was used to train a classifier model to be used with male patients. In embodiments, the gender of the patient is used to select the classifier model. In embodiments, training data comprises a greater number of patients without cancer than with cancer, wherein training of the classifier models comprises reprocessing the training data by using a stratified sampling technique to improve selection of negative samples. In embodiments, the classifier model has a performance of a Receiver Operator Characteristic (ROC) curve with a sensitivity value of at least 0.8 and a specificity value of at least 0.8; or optionally a sensitivity value of at least 0.9 or 0.95 and a specificity value of at least 0.9 or 0.95. In embodiments, the PPV is measured in each of the risk categories such as mild, moderate and high-risk categories. In certain embodiments, the performance of the present classifier model as measured by PPV is less than 2% for mild category; less than 5% for the moderate category and less than 12% for the high-risk category. See Example 2.

In embodiments, the machine learning system generates a classifier model that may be static. In other words, the classifier model is trained and then its use is implemented with a computer implemented system wherein patient data (e.g., biomarker marker measurements and age) are input and the classifier model provides an output that is used to classify patients.

In other embodiments, the classifier models are continuously, or routinely, being updated and improved wherein the input values, output values, along with a diagnostic indicator, type of cancer and/or time to diagnosis from patients are used to further train the classifier models. In embodiments, the classifier model is selected based on gender and trained using age and measurement of AFP, CEA, CA19-9, CYFRA21-1, SCC, and PSA (for men) or AFP, CEA, CA19-9, CYFRA21-1, SCC, CA125, and CA15-3 (for women).

In embodiments provided herein is a classifier model to predict an increased risk of having or developing cancer, for an asymptomatic patient. In embodiments, this first classifier model is generated by a machine learning system using training data that comprises values of a panel of at least CEA and AFP biomarkers, age, and a diagnostic indicator, for a population of patients. In embodiments, the first classifier model was trained using data from only a male cohort or a female cohort.

In embodiments, the first classifier model assigns a risk score of having or developing cancer to the patient, wherein the risk score is generated using a first classifier model using input variables of measured values of AFP, CEA, CA19-9, CYFRA21-1, SCC, and PSA biomarkers (for men) or AFP, CEA, CA19-9, CYFRA21-1, SCC, CA125, and CA15-3 biomarkers (for women) and optionally age, when an output of the first classifier model is a numerical expression of the percent likelihood of having or developing cancer. In embodiments, the classifier model classifies the patient into a risk category of having or developing cancer using the assigned risk score, wherein a risk score percent likelihood of having or developing cancer is greater than the percent prevalence of cancer in the population is deemed an increased risk category. In exemplary embodiments, the output is a probability value, wherein the threshold is set to separate patients into a low or non-risk category (those patients wherein their risk is no more than the population reflective of the training data) from an increased risk category (those patients with an increased risk of having or developing cancer as compared to a population reflective of the training data). In certain embodiments, the increased risk category may be further subdivided, such as a mild, moderate and/or high-risk category.

In embodiments, the assigned a risk score is presented as a percent, e.g., X of 100, or multiplier number. In certain embodiments, a patient may be assigned a 2 to 10% risk score (of having or developing cancer) wherein the incidence of cancer in the population used to train the classifier model is about 1%. In embodiments, those percentage risk scores may be presented as X of 100, e.g., 3 out of 100 wherein a patient with that score has an approximately 3 out of 100 risk of developing cancer within one year from when the biomarkers were measured. In this instance, a threshold cut off, wherein a risk score at or below would be considered normal, and a risk score above would be considered an increased risk. In certain embodiments, the threshold cut off value may be 1 out of 100, corresponding to a “normal” risk of having cancer in a heterogenous population of 1%. In other embodiments, the threshold cut off value may be 2 out of 100, corresponding to a “normal” risk of having cancer in a heterogenous population of 2%. In certain embodiments, the threshold cut off value may be 3 out of 100, corresponding to a “normal” risk of having cancer in a heterogenous population of 3%.

In certain other embodiments, the patient may be assigned a multiplier number. In embodiments, the risk score is not an output value, but a value assigned to a risk category, such as an increased risk category, wherein the output value is used to classify a patient into the risk category. In certain embodiments, an output value is a predicted probability value that may range from 0 to 1, wherein that value is used to classify a patient into a risk category. The risk score assigned to a risk category is then calculated by comparing the predicted probability assigned to a risk category to the prevalence of cancer in a population. In embodiments, a patient may have an increased risk of having or developing cancer selected from the group consisting of: bile duct cancer, bone cancer, colon cancer, colorectal cancer, gallbladder cancer, kidney cancer, liver or hepatocellular cancer, lobular carcinoma, lung cancer, melanoma, ovarian cancer, pancreatic cancer, prostate cancer, skin cancer, and testicular cancer.

Disclosed herein is a machine learning system comprising at least one processor for predicting an increased risk for cancer. In certain embodiments, the processor is configured to obtain measured values of a panel of biomarkers in a sample from a patient, wherein a value of a biomarker corresponds to a level of the biomarker in the sample, obtain clinical parameters from the patient including age and gender, and generate a first classifier model by the machine learning system to classify the patient into a risk category of having or developing cancer based on an assigned risk score, wherein the first classifier model classifies a patient into an increased risk category when the output of the first classifier model is greater than a threshold, and wherein the first classifier model is generated by the machine learning system using training data that comprises values from a panel of at least two biomarkers, age, gender and a diagnostic indicator for a population of patients. In embodiments, the training data is from longitudinal study wherein the biomarker measurements are obtained months, or years, before a cancer diagnosis is confirmed (or not) for a patient in the training data cohort. In embodiments, the threshold is the known prevalence of cancer in the population.

In embodiments, the first classifier model comprises a support vector machine, a decision tree, a random forest, a neural network, a deep learning neural network, or a logistic regression algorithm.

In embodiments provided herein is a second classifier model for identification of likely cancer systems for those classified in a risk category. Disclosed herein is a second classifier model to predict at least one most likely organ system malignancy and/or the top three most likely cancer system and/or a specific cancer. In certain embodiments, the second classifier model is applied to patients that are classified into an increased risk category for having or developing cancer. As with the first classifier model, the second classifier model was trained with measured biomarkers from a longitudinal study, and age, wherein one classifier model was trained from and for female patients and another classifier model was trained from and for male patients.

In embodiments, the second classifier model was generated by a machine learning system using training data that comprises values from a panel of at least two biomarkers, age, and a diagnostic indicator for a population of patients. In embodiments, the second classifier model was trained using data from only a male cohort or only a female cohort. In embodiments, the training data comprises values of a panel of at least six biomarkers. In embodiments, the training data comprises values from a panel of biomarkers selected from AFP, CEA, CA125, CA19-9, CA 15-3, CYFRA21-1, PSA and SCC.

In exemplary embodiments, a second classifier model is generated by a machine learning system using training data that comprises a male cohort only, values of a panel of six biomarkers comprising AFP, CEA, CA19-9, CYFRA21-1, PSA and SCC, and age. In other exemplary embodiments, a second classifier model is generated by a machine learning system using training data that comprises a female cohort only, values of a panel of seven biomarkers comprising AFP, CEA, CA125, CA19-9, CA 15-3, CYFRA21-1 and SCC, and age.

In embodiments, the second classifier model assigns a patient into an organ system class membership using input variables of age and the measured values of the panel of biomarkers from the patient. See Table A. In certain embodiments, the second classifier model assigns a patient into a specific cancer class membership using input variables of age and the measured values of the panel of biomarkers from the patient. In embodiments, the class membership is for an organ system selected from genitourinary (GU), gastrointestinal (GI), pulmonary, dermatological, hematological, nervous system, gynecological, or general. In certain embodiments, the class membership is for an organ system selected from general surgery (breast cancer, thyroid cancer or liposarcoma), chest (lung cancer), dermatology (skin cancer), ear, nose and throat (head & neck cancer, parotid cancer), gastrointestinal (GI) (HCC, CRC, Gastric cancer, Pancreatic cancer, Esophageal cancer, Gallbladder cancer), genitourinary (GU) (bladder cancer, prostrate cancer and RCC), hematology (leukemia and lymphoma), neurology (CNS cancer) and gynecological (cervical cancer, ovarian cancer and uteral cancer). In certain embodiments, the class membership is for a cancer selected from breast cancer, bile duct cancer, bone cancer, cervical cancer, colon cancer, colorectal cancer, gallbladder cancer, kidney cancer, liver or hepatocellular cancer, lobular carcinoma, lung cancer, melanoma, ovarian cancer, pancreatic cancer, prostate cancer, skin cancer, or testicular cancer.

In embodiments, the second classifier model is selected based on the gender of the patient. In embodiments, the input variables for a male patient comprises measured values from a panel of at least six biomarkers and age. In embodiments, the panel of biomarkers is selected from AFP, CEA, CA125, CA19-9, CA 15-3, CYFRA21-1, PSA and SCC. In exemplary embodiments, the input variable for a male patient comprises measured values from AFP, CEA, CA19-9, CYFRA21-1, PSA and SCC, and age. In other embodiments, the input variables for a female patient comprises measured values from a panel of at least six biomarkers and age. In exemplary embodiments, the input variables for a female patent comprises measured values from AFP, CEA, CA125, CA19-9, CA 15-3, CYFRA21-1 and SCC, and age.

In embodiments, the second classifier model comprises a pattern recognition algorithm. In exemplary embodiments, the second classifier model comprises k-Nearest Neighbors algorithm (kNN). In certain embodiments, the second classifier model comprises a support vector machine, a decision tree, a random forest, a neural network, a deep learning neural network, or a logistic regression algorithm.

In embodiments provided herein is a third classifier model for classifying subjects into a category with a time range to be re-tested based on the time to diagnosis data of the present study. See FIGS. 3 and 4. In certain embodiments, the first classifier model is used to obtain a risk score for the patient wherein the patients are further classified in a follow-up category using the risk score generated by the first classifier model, wherein a second pre-determined threshold, and optionally third pre-determined threshold, separate the follow-up categories and the second, and optionally third, pre-determined threshold is a median time to definitive diagnosis following measurement of the biomarkers of the population of patients. In embodiments, the output of the classifier model is provided to a user as the patient risk score and follow-up category, wherein follow-up testing is selected from repeat testing in about one year, repeat testing in less than about 1 year and/or confirmatory cancer diagnostic testing.

In embodiments, the third classifier model is a computer-implemented method for generating a follow-up cancer diagnostic testing classifier model, wherein the classifier model is generated using the first classifier model and median time to definitive diagnosis in each risk group from longitudinal or retrospective data. In embodiments the third classifier model is generated comprising the following steps, a) obtaining, by one or more processors, a data set from a population of patients comprising a risk score of having or developing cancer and time to definitive diagnosis following measurement of one or more biomarkers, wherein the risk score is generated by a first classifier model using inputs of measured values of the one or more biomarkers, optionally age, and a diagnostic indicator, from a population of patients); b) segmenting the data into two or more groups based on the risk score; c) determining a median time to definitive diagnosis in each group; and, d) generating the classifier model for follow-up cancer diagnostic testing based on the correlation between the risk score and the time to definitive diagnosis, wherein the classifier model provides output selected from repeat testing in about one year, repeat testing in less than about 1 year and/or confirmatory cancer diagnostic testing.

Measuring Biomarkers in a Sample

As part of the present method, a panel of markers from an asymptomatic human subject may be measured. There are many methods known in the art for measuring either gene expression (e.g., mRNA) or the resulting gene products (e.g., polypeptides or proteins) that can be used in the present methods, and known to one of skill in the art. However, for at least 2-3 decades tumor antigens (e.g. CEA, and AFP.) have been the most widely utilized biomarkers for cancer detection throughout the world and are the preferred tumor marker type for the present invention.

For tumor antigen detection, testing is preferably conducted using an automated immunoassay analyzer from a company with a large installed base. Representative analyzers include the Elecsys® system from Roche Diagnostics or the Architect® Analyzer from Abbott Diagnostics. Using such standardized platforms permits the results from one laboratory or hospital to be transferable to other laboratories around the world. However, the methods provided herein are not limited to any one assay format or to any particular set of markers that comprise a panel. For example, PCT International Pat. Pub. No. WO 2009/006323; US Pub. No. 2012/0071334; US Pat. Pub. No. 2008/0160546; US Pat. Pub. No. 2008/0133141; US Pat. Pub. No. 2007/0178504 (each herein incorporated by reference) teaches a multiplex lung cancer assay using beads as the solid phase and fluorescence or color as the reporter in an immunoassay format. Hence, the degree of fluorescence or color can be provided in the form of a qualitative score as compared to an actual quantitative value of reporter presence and amount.

For example, the presence and quantification of one or more antigens or antibodies in a test sample can be determined using one or more immunoassays that are known in the art. Immunoassays typically comprise: (a) providing an antibody (or antigen) that specifically binds to the biomarker (namely, an antigen or an antibody); (b) contacting a test sample with the antibody or antigen; and (c) detecting the presence of a complex of the antibody bound to the antigen in the test sample or a complex of the antigen bound to the antibody in the test sample.

Well-known immunological binding assays include, for example, an enzyme linked immunosorbent assay (ELISA), which is also known as a “sandwich assay”, an enzyme immunoassay (EIA), a radioimmunoassay (MA), a fluoroimmunoassay (FIA), a chemiluminescent immunoassay (CLIA), a counting immunoassay (CIA), a filter media enzyme immunoassay (META), a fluorescence-linked immunosorbent assay (FLISA), agglutination immunoassays and multiplex fluorescent immunoassays (such as the Luminex Lab MAP), immunohistochemistry, etc. For a review of the general immunoassays, see also, Methods in Cell Biology: Antibodies in Cell Biology, volume 37 (Asai, ed. 1993); Basic and Clinical Immunology (Daniel P. Stites; 1991).

The immunoassay can be used to determine a test amount of an antigen in a sample from a subject. First, a test amount of an antigen in a sample can be detected using the immunoassay methods described above. If an antigen is present in the sample, it will form an antibody-antigen complex with an antibody that specifically binds the antigen under suitable incubation conditions as described herein. The amount, activity, or concentration, etc. of an antibody-antigen complex can be determined by comparing the measured value to a standard or control. The AUC for the antigen can then be calculated using techniques known, such as, but not limited to, a ROC analysis.

In another embodiment, gene expression of markers (e.g., mRNA) is measured in a sample from a human subject. For example, gene expression profiling methods for use with paraffin-embedded tissue include quantitative reverse transcriptase polymerase chain reaction (qRT-PCR), however, other technology platforms, including mass spectroscopy and DNA microarrays can also be used. These methods include, but are not limited to, PCR, Microarrays, Serial Analysis of Gene Expression (SAGE), and Gene Expression Analysis by Massively Parallel Signature Sequencing (MPSS).

Any methodology that provides for the measurement of a marker or panel of markers from a human subject is contemplated for use with the present methods. In certain embodiments, the sample from the human subject is a tissue section such as from a biopsy. In another embodiment, the sample from the human subject is a bodily fluid such as blood, serum, plasma or a part or fraction thereof. In other embodiments, the sample is a blood or serum and the markers are proteins measured therefrom. In yet another embodiment, the sample is a tissue section and the markers are mRNA expressed therein. Many other combinations of sample forms from the human subjects and the form of the markers are contemplated.

Many markers are known for diseases, including cancers and a known panel can be selected, or as was done by the present Applicants, a panel can be selected based on measurement of individual markers in longitudinal clinical samples wherein a panel is generated based on empirical data for a desired disease such as cancer.

Examples of biomarkers that can be employed include molecules detectable, for example, in a body fluid sample, such as, antibodies, antigens, small molecules, proteins, hormones, enzymes, genes and so on. However, the use of tumor antigens has many advantages due to their widespread use over many years and the fact that validated and standardized detection kits are available for many of them for use with the aforementioned automated immunoassay platforms.

In embodiments, the biomarkers are selected from AFP, and CEA. In certain embodiments, additional markers may be selected from markers associated with a cancer selected from bile duct cancer, bone cancer, pancreatic cancer, cervical cancer, colon cancer, colorectal cancer, gallbladder cancer, liver or hepatocellular cancer, ovarian cancer, testicular cancer, lobular carcinoma, prostate cancer, and skin cancer or melanoma. In other embodiments, a panel of markers comprises markers associated with breast cancer. In certain embodiment, a panel of biomarkers comprises markers associated with “pan cancer”.

In certain regions of the world, most notably in the Far East, many hospitals and “Health Check Centers” offer panels of tumor markers to patients as part of their annual physicals or check-ups. These panels are offered to patients without noticeable signs or symptoms of, or predisposition to, any particular cancer and are not specific to any one tumor type (i.e. “pan-cancer”). Exemplary of such testing approaches is the one reported by Y.-H. Wen et al., Clinica Chimica Acta 450 (2015) 273-276, “Cancer Screening Through a Multi-Analyte Serum Biomarker Panel During Health Check-Up Examinations: Results from a 12-year Experience.” The authors report on the results from over 40,000 patients tested at their hospital in Taiwan between 2001 and 2012. The patients were tested with the following biomarkers: AFP, CA 15-3, CA125, PSA, SCC, CEA, CA 19-9, and CYFRA, 21-1 using kits available from Roche Diagnostics, Abbott Diagnostics, and Siemens Healthcare Diagnostics. The sensitivity of the panel for identifying the four most commonly diagnosed malignancies in that region (i.e., liver cancer, lung cancer, prostate cancer, and colorectal cancer) was 90.9%, 75.0%, 100% and 76%, respectively. Subjects with at least one of the markers showing values above the cut-off point were considered positive for the assay. No algorithm was reported. Moreover, neither clinical parameters nor biomarker velocity were factored in with this test.

It is believed that the methods and machine learning systems according to the present invention can improve and enhance the pan-cancer biomarker panel reported by the Taiwanese group and readily permit its use in other parts of the world. For example, an algorithm that combines biomarker values with clinical parameters could be employed that automatically improves using the machine learning software.

A panel can comprise any number of markers as a design choice, seeking, for example, to maximize specificity or sensitivity of the classifier model. Hence, the present methods may ask for presence of at least one of two or more biomarkers, three or more biomarkers, four or more biomarkers, five or more biomarkers, six or more biomarkers, seven or more biomarkers, eight biomarkers or more as a design choice.

Thus, in one embodiment, the panel of biomarkers may comprise at least two, at least three, at least four, at least five, at least six, at least seven, at least eight, at least nine or at least ten or more different markers. In one embodiment, the panel of biomarkers comprises about two to ten different markers. In another embodiment, the panel of biomarkers comprises about four to eight different markers. In yet another embodiment, the panel of markers comprises about six or about seven different markers.

Generally, a sample is committed to the assay and the results can be a range of numbers reflecting the presence and level (e.g., concentration, amount, activity, etc.) of presence of each of the biomarkers of the panel in the sample.

The choice of the markers may be based on the understanding that each marker, when measured and normalized, contributed equally as an input variable for the classifier model. Thus, in certain embodiments, each marker in the panel is measured and normalized wherein none of the markers are given any specific weight. In this instance each marker has a weight of 1.

In other embodiments, the choice of the markers may be based on the understanding that each marker, when measured and normalized, contributed unequally as an input variable for the classifier model. In this instance, a particular marker in the panel can either be weighted as a fraction of 1 (for example if the relative contribution is low), a multiple of 1 (for example if the relative contribution is high) or as 1 (for example when the relative contribution is neutral compared to the other markers in the panel).

In still other embodiments, a machine learning system may analyze values from biomarker panels without normalization of the values. Thus, the raw value obtained from the instrumentation to make the measurement may be analyzed directly.

The use in a clinical setting of the embodiments presented herein are now described in the context of “pan cancer” and specific cancer screening.

Primary care healthcare practitioners, who may include physicians specializing in internal medicine or family practice as well as physician assistants and nurse practitioners, are among the users of the techniques disclosed herein. These primary care providers typically see a large volume of patients each day. In one instance these patients are at risk for lung cancer due to smoking history, age, and other lifestyle factors. In 2012 about 18% of the U.S. population was current smokers and many more were former smokers with a lung cancer risk profile above that of a population that has never smoked.

A blood sample from patient, such as a patient 50 years of age or older, is sent to a laboratory qualified to test the sample using a panel of biomarkers, such as those used to train the present classifier models generated by a machine learning system. Non-limiting lists of such biomarkers are herein included throughout the specification including the examples. In lieu of blood, other suitable bodily fluids such a sputum or saliva might also be utilized.

The measured values of the biomarkers are then used as input values, along with age, to be used with the first classifier model in a computer implemented system. An output value is obtained and compared to a threshold value wherein the threshold is empirically determined and set to separate patients in a low risk category from those in an increased risk for having or developing cancer. The threshold value is empirically determined using longitudinal clinical data. If the risk calculation is to be made at the point of care, rather than at the laboratory, a software application compatible with mobile devices (e.g., a tablet or smart phone) may be employed.

For those patients classified into an increased risk category, the input variables of measured biomarkers and age may be used with the second classifier model in a computer implemented system. An output value is obtained and compared to the longitudinal clinical data used to train the second classifier model and assigned a class membership, wherein the class memberships are organ system. In certain embodiments, the class membership is further defined by a specific cancer type, e.g., lung cancer.

Once the physician or healthcare practitioner has a risk score for the patient (i.e. risk that the patient has or will develop cancer relative to a population of others with comparable epidemiological factors) and the most likely organ malignancy or specific cancer, follow-up testing can be recommended for those at higher risk, such as radiography screening or tissue biopsy. It should be appreciated that the precise numerical cut off above which further testing is recommended may vary depending on many factors including, without limitation, (i) the desires of the patients and their overall health and family history, (ii) practice guidelines established by medical boards or recommended by scientific organizations, (iii) the physician's own practice preferences, and (iv) the nature of the biomarker test including its overall accuracy and strength of validation data.

It is believed that use of the embodiments presented herein will have the twin benefits of ensuring that the most at-risk patients undergo further diagnostic testing so as to detect early tumors and occult cancer that can be cured with surgery while reducing the expense and burden of false positives associated with stand-alone screening.

Embodiments of the present invention further provide for an apparatus for assessing a subject's risk level for the presence of cancer and correlating the risk level with an increase or decrease of the presence of cancer after testing relative to a population or a cohort population. The apparatus may comprise a processor configured to execute computer readable media instructions (e.g., a computer program or software application, e.g., a machine learning system, to receive the concentration values from the evaluation of biomarkers in a sample and, in combination with other risk factors (e.g., medical history of the patient, publicly available sources of information pertaining to a risk of developing cancer, etc.) may determine a risk score and compare it to a grouping of stratified cohort population comprising multiple risk categories.

The apparatus can take any of a variety of forms, for example, a handheld device, a tablet, or any other type of computer or electronic device. The apparatus may also comprise a processor configured to execute instructions (e.g., a computer software product, an application for a handheld device, a handheld device configured to perform the method, a world-wide-web (WWW) page or other cloud or network accessible location, or any computing device. In other embodiments, the apparatus may include a handheld device, a tablet, or any other type of computer or electronic device for accessing a machine learning system provided as a software as a service (SaaS) deployment. Accordingly, the correlation may be displayed as a graphical representation, which, in some embodiments, is stored in a database or memory, such as a random access memory, read-only memory, disk, virtual memory, etc. Other suitable representations, or exemplifications known in the art may also be used.

The apparatus may further comprise a storage means for storing the correlation, an input means, and a display means for displaying the status of the subject in terms of the particular medical condition. The storage means can be, for example, random access memory, read-only memory, a cache, a buffer, a disk, virtual memory, or a database. The input means can be, for example, a keypad, a keyboard, stored data, a touch screen, a voice-activated system, a downloadable program, downloadable data, a digital interface, a hand-held device, or an infrared signal device. The display means can be, for example, a computer monitor, a cathode ray tube (CRT), a digital screen, a light-emitting diode (LED), a liquid crystal display (LCD), an X-ray, a compressed digitized image, a video image, or a hand-held device. The apparatus can further comprise or communicate with a database, wherein the database stores the correlation of factors and is accessible to the user.

In another embodiment of the present invention, the apparatus is a computing device, for example, in the form of a computer or hand-held device that includes a processing unit, memory, and storage. The computing device can include or have access to a computing environment that comprises a variety of computer-readable media, such as volatile memory and non-volatile memory, removable storage and/or non-removable storage. Computer storage includes, for example, RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output, and/or a communication connection. The input can be one or several devices, such as a keyboard, mouse, touch screen, or stylus. The output can also be one or several devices, such as a video display, a printer, an audio output device, a touch stimulation output device, or a screen reading output device. If desired, the computing device can be configured to operate in a networked environment using a communication connection to connect to one or more remote computers. The communication connection can be, for example, a Local Area Network (LAN), a Wide Area Network (WAN) or other networks and can operate over the cloud, a wired network, wireless radio frequency network, and/or an infrared network.

Artificial intelligence systems include computer systems configured to perform tasks usually accomplished by humans, e.g., speech recognition, decision making, language translation, image processing and recognition, etc. In general, artificial intelligence systems have the capacity to learn, to maintain and access a large repository of information, to perform reasoning and analysis in order to make decisions, as well as the ability to self-correct.

Artificial intelligence systems may include knowledge representation systems and machine learning systems. Knowledge representation systems generally provide structure to capture and encode information used to support decision making. Machine learning systems are capable of analyzing data to identify new trends and patterns in the data. For example, machine learning systems may include neural networks, induction algorithms, genetic algorithms, etc. and may derive solutions by analyzing patterns in data.

In certain embodiments, the present classifier models comprise an algorithm such as a support vector machine, a decision tree, a random forest, a neural network, a deep learning neural network, a logistic regression or a pattern recognition algorithm. The present classifier models may be used to classify an individual patient into one of a plurality of categories, e.g., a category indicative of a likelihood of cancer or a category indicating that cancer is not likely. Inputs to the classifier model may include a panel of biomarkers associated with the presence of cancer as well as clinical parameters. In certain embodiments, clinical parameters include one or more of the following: (1) age; (2) gender; (3) smoking history in years; (4) number of packs per year; (5) symptoms; (6) family history of cancer; (7) concomitant illnesses; (8) number of nodules; (9) size of nodules; and (10) imaging data and so forth. In exemplary embodiments, the clinical parameter used as in put value is age wherein gender is used to train the classifier model providing a classifier model for male patients and a separate classifier model for female patients.

In embodiments, a Logistic Regression Model can be used to generate a present classifier model. Logistic regression is a simple but powerful method, especially for binary outcome. One key component is the logistic function, which could convert the multivariable input into the probability of the outcome between 0 and 1. Among all the machine learning algorithms, logistic regression has multiple advantages. First, no assumption is needed such as normal distribution of independent variables; Second, no assumption is needed about linear relationship between outcome and covariates. Most importantly, it is easy to understand and interpret the results.

In certain embodiments a Support Vector Machines (SVM) Model can be used to generate a present classifier model. SVM is another popular machine learning algorithm based on statistical learning theory. The SVM algorithm is used to find a decision boundary which could maximize the distance between the two closest classes. The biggest advantage for SVM is that it could model non-linear decision boundary. It has multiple kernel functions and is generally robust against over fitting. However, one disadvantage to this algorithm is that SVM is very memory intensive and may not scale well to large datasets.

In embodiments, a Random Forest Model can be used to generate a present classifier model. Random forests are considered as one of the most accurate machine learning methods, which are an ensemble classifier and proved to be the top winner in several data competitions. Random forests consist of many decision trees and combine the result from the individual trees. The attractive benefits using random forests lie in the following facts: 1) random forests could handle thousands of input variables without variable selection, which is heavy burden for logistic regression; 2) through large number of decision trees within random forest, it could produce an unbiased estimate of the generalization error; 3) it may allow large portion of missing data. We followed the general procedures for optimizing hyperparameters in Random Forest classification. The two key hyperparameters are number of trees (ntree) and number of variables randomly sampled as candidates at each split (mtry). We used the R package ‘Caret’ to support our optimization process. We created a list of different combination of mtry (from 1 to 8) and ntree=(from 50 to 1000 by 50), and compared the performance, and determined the best ntree and mtry. For the male model, ntree=500, mtry=3, for the female model, ntree=800, mtry=3.

A variety of machine learning models are available, including support vector machines, decision trees, random forests, neural networks or deep learning neural networks. Generally, support vector machines (SVMs) are supervised learning models that analyze data for classification and regression analysis. SVMs may plot a collection of data points in n-dimensional space (e.g., where n is the number of biomarkers and clinical parameters), and classification is performed by finding a hyperplane that can separate the collection of data points into classes. In some embodiments, hyperplanes are linear, while in other embodiments, hyperplanes are non-linear. SVMs are effective in high dimensional spaces, are effective in cases in which the number of dimensions is higher than the number of data points, and generally work well on data sets with clear margins of separation.

Decision trees are a type of supervised learning algorithm also used in classification problems. Decision trees may be used to identify the most significant variable that provides the best homogenous sets of data. Decision trees split groups of data points into one or more subsets, and then may split each subset into one or more additional categories, and so forth until forming terminal nodes (e.g., nodes that do not split). Various algorithms may be used to decide where a split occurs, including a Gini Index (a type of binary split), Chi-Square, Information Gain, or Reduction in Variance. Decision trees have the capability to rapidly identify the most significant variables among a large number of variables, as well as identify relationships between two or more variables. Additionally, decision trees can handle both numerical and non-numerical data. This technique is generally considered to be a non-parametric approach, e.g., the data does not have to fit a normal distribution.

Random forest (or random decision forest) is a suitable approach for both classification and regression. In some embodiments, the random forest method constructs a collection of decision trees with controlled variance. Generally, for M input variables, a number of variables (nvar) less than M is used to split groups of data points. The best split is selected and the process is repeated until reaching a terminal node. Random forest is particularly suited to process a large number of input variables (e.g., thousands) to identify the most significant variables. Random forest is also effective for estimating missing data.

Neural nets (also referred to as artificial neural nets (ANNs)) are described throughout this application. A neural net, which is a non-deterministic machine learning technique, utilizes one or more layers of hidden nodes to compute outputs. Inputs are selected and weights are assigned to each input. Training data is used to train the neural networks, and the inputs and weights are adjusted until reaching specified metrics, e.g., a suitable specificity and sensitivity.

ANNs may be used to classify data in cases in which correlation between dependent and independent variables is not linear or in which classification cannot be easily performed using an equation. More than 25 different types of ANNs exist, with each ANN yielding different results based on different training algorithms, activation/transfer functions, number of hidden layers, etc. In some embodiments, more than 15 types of transfer functions are available for use with the neural network. Prediction of the likelihood of having cancer is based upon one or more of the type of ANN, the activation/transfer function, the number of hidden layers, the number of neurons/nodes, and other customizable parameters.

Deep learning neural networks, another machine learning technique, are similar to regular neural nets, but are more complex (e.g., typically have multiple hidden layers) and are capable of automatically performing operations (e.g., feature extraction) in an automated manner, generally requiring less interaction with a user than a traditional neural net.

In preferred embodiments, this disclosure provides the following aspects:
1. A method for identifying a patient for follow-up cancer diagnostic testing, the method comprising:
   a) assigning a risk score of having or developing cancer to the patient, wherein the risk score is generated using a first classifier model using input variables of measured values of a panel of biomarkers from the patient and clinical factors including at least age and a diagnostic indicator, for a population of patients, when an output of the first classifier model is a numerical expression of the percent likelihood of having or developing cancer;
   b) classifying the patient into an increased risk category of having or developing cancer when their risk score, generated by the first classifier model, is above a first pre-determined threshold, wherein the first pre-determined threshold is a prevalence of cancer in the population of patients;
   c) classifying those patients in the increased risk category into a follow-up category using the risk score generated by the first classifier model, wherein a second pre-determined threshold, and optionally third pre-determined threshold, separate the follow-up categories and the second, and optionally third, pre-determined threshold is a median time to definitive diagnosis following measurement of the biomarkers of the population of patients; and,
   d) providing a notification to a user of the patient risk score and follow-up category, wherein follow-up testing is selected from repeat testing in about one year, repeat testing in less than about 1 year and/or confirmatory cancer diagnostic testing.
2. A computer-implemented method for generating a follow-up cancer diagnostic testing classifier model comprising:
   a) obtaining, by one or more processors, a data set from a population of patients comprising a risk score of having or developing cancer and time to definitive diagnosis following measurement of one or more biomarkers, wherein the risk score is generated by a first classifier model using inputs of measured values of the one or more biomarkers, optionally age, and a diagnostic indicator, from a population of patients;
   b) segmenting the data into two or more groups based on the risk score; and,
   c) determining a median time to definitive diagnosis in each group; and,
   d) generating the classifier model for follow-up cancer diagnostic testing based on the correlation between the risk score and the time to definitive diagnosis, wherein the classifier model provides output selected from repeat testing in about one year, repeat testing in less than about 1 year and/or confirmatory cancer diagnostic testing.
3. A method for screening for cancers in an asymptomatic (i.e., the person is not exhibiting any symptoms or other signs of cancer (e.g., biomarker (BM) or tumor marker (TM) levels, imaging results, pain, etc.) human subject comprising:
   a) obtaining a first blood sample from the human subject;
   b) measuring a panel of at least two markers in the sample, wherein said markers are selected from the group consisting of CEA, AFP, CA125, CA15-3 CA19-9, Cyfra, and PSA;
   c) providing machine learning software to produce a cancer likelihood score (e.g., a predictive value within a time period of having or developing cancer), wherein said software is built from data from individuals previously tested with said marker panel and for which cancer outcomes (e.g., cancer is known and has been diagnosed as present in those individuals, or individuals have been diagnosed to be cancer free) are known;
   d) generating a cancer likelihood score for the human subject;
   e) using said cancer likelihood score to calculate the optimal time interval when said at least two markers should be re-measured in a second blood sample from the human subject;
   f) obtaining a second blood sample from the human subject based on said time interval; and
   g) re-measuring said panel of markers in the second blood sample and comparing the changes in marker levels between the first and second blood samples.
   h)
      Other aspects of this disclosure are also contemplated as will be understood by those of ordinary skill in the art.

### EXAMPLES

The Examples below are given so as to illustrate the practice of this invention. They are not intended to limit or define the entire scope of this invention.

### Example 1: Material and Methods

Patient Eligibility

The study was approved by the Institutional Review Board of Chang Gung Medical Foundation (No. 201601798B0). We followed the Standards for Reporting of Diagnostic Accuracy 2015 [19]. Patient records were anonymized and de-identified prior to analysis. 27,938 (12,622 men and 15,316 women) apparently asymptomatic individuals were included who had at least one voluntary test with a panel of tumor markers between May 2001 and April 2015 at the Linkou or Kaohsiung branch of CGMH. All individuals had complete data on 6 tumor markers (AFP, CEA, CA19-9, CYFRA21-1, SCC, and PSA) for men and 7 tumor markers (AFP, CEA, CA19-9, CYFRA21-1, SCC, CA125, and CA15-3) for women. AFP, CEA, CA19-9, SCC, PSA, CA125, and CA15-3 were measured using commercially available kits (Abbott Diagnostics, Abbott Park, Ill., USA). CYFRA21-1 was analytically determined with a commercially available kit (Roche Diagnostics Corp., Indianapolis, Ind., USA). All assays met the requirements of the College of American Pathologists (CAP) Laboratory Accreditation Program, ensuring the results were accurate and reproducible. All the cases were tracked for at least 12 months subsequent to testing. Cancer diagnoses were obtained from the Taiwan Cancer Registry of the Ministry of Health and Welfare to determine whether each patient had received a new diagnosis of cancer subsequent to tumor marker testing. Of the 12,622 men, 186 received a new diagnosis of cancer. Similarly, of the 15,316 women, 156 received a new diagnosis of cancer. The data from CGMH Linkou branch, included 8415 men (124 cancers) and 10,211 women (108 cancers). In the dataset from CGMH Kaohsiung branch, 62 out of 4207 men and 52 out of 5105 women received a new diagnosis of cancer.

Training, Validating, and Testing Cancers Screening Machine Learning Models

Data from CGMH Linkou branch was used to train ML models and data from CGMH Kaohsiung branch was used as the independent third-party testing dataset for validating both the male and female models. The input features included the tumor marker values, age, and gender. Since the sourced data is real word data (RWD), the datasets were extremely imbalanced, with the ratio of cancer cases to non-cancer cases around 1:100. Given this imbalance, a random subsampling approach was applied to build the model. In general, classical machine learning algorithms such as support vector machine (SVM) and random forest (RF) are robust enough to cope with imbalanced data. However, when the data are extremely imbalanced (e.g., around 1:100 in the study), additional techniques should be adopted to improve the classification performance. For example, SVMs minimize the error over the entire dataset in order to generate these models, so they are biased towards the majority class when the imbalance is severe [20]. RF induces each constituent tree from a bootstrap sample of the training data [21]. When using an extremely imbalanced dataset, there is a significant probability that a bootstrap sample contains few or even none of the minority class, resulting in a tree with poor performance for predicting the minority class [22]. Subsampling of the majority group is a well-known technique to deal with extremely imbalanced datasets. The subsampling method is simple and not inferior to other methods in mitigating data imbalance [22]. Moreover, subsampling uses real world data and does not create artificial data like other oversampling methods.

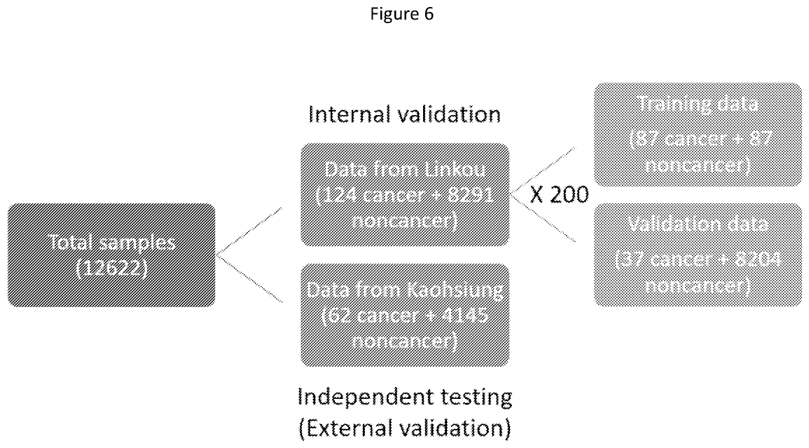

Subsampling was repeated 200 times and internally cross validated the ML models based on the average area under the receiver operating characteristic curve (AUROC), sensitivity, and specificity. The internal cross validation was conducted by using partial data from CGMH Linkou branch to train ML models and using the other partial data from CGMH Linkou branch (unseen in the training process) to validate the ML models. ML models trained by the data from CGMH Linkou branch were validated by using the independent third-party testing dataset from CGMH Kaohsiung branch. ML algorithms including logistic regression (LR), random forest (RF), and support vector machine (SVM) were used. Cutoffs of ML models were determined based on the Youden index of the receiver operator characteristic (ROC) curves by the data from CGMH Linkou branch (training dataset). See FIG. 6. The cutoffs were used to classify low versus non-low risk groups. Furthermore, the non-low risk group was further equally divided into mild, moderate, and high-risk groups based on their predictive probabilities. The aim of subgrouping was to correlate the risk-stratification to the clinical prognosis but not keep these groups equal. The cutoffs (e.g. predetermined threshold) were determined by the following two steps: (1) determining the cutoff (cutoff No. 1) that discriminates the low risk group from the non-low risk group; (2) based on cutoff No. 1, we determined cutoff No. 2, cutoff No. 3, and cutoff No. 4 for stratifying the mild, moderate, and high-risk groups based on their predictive probabilities. Cutoff No. 2 and No. 3 were determined intuitively by linearly dividing the probability. The cutoffs are illustrated in FIG. 7.

Organ System for Localizing Tissue Origin

A second algorithm was developed to suggest the most likely organ system from which a cancer has originated in order to simplify the follow-up processes when an at-risk case is identified by the cancer screening algorithms. Each organ system label included several different cancer types, and all the cancer types within an organ system label are highly related (Table A). Moreover, all the cancer types within an organ system label are usually treated by the same medical specialist. The model used the top-N nearest-neighbor method for the analysis of tumor tissue origin based on organ systems. N was determined to be 10 in the preliminary trial. For the prediction of tissue origin, the 10 nearest cases were clustered based on the organ system label. The percentage of each organ system label was calculated in these 10 cases. The percentage of each organ system was used to rank the most likely (top N) organ systems. The second algorithm was based on the tumor marker feature space. Model performance was evaluated by repeated 5-fold cross validation for 10 times.

TABLE A
Organ system clustering and labelling

| Organ System | | |
|---|---|---|
| Label | Female | Male |
| General Surgery | Breast cancer, Thyroid | NA |
| | cancer, and Liposarcoma | |
| Chest | Lung cancer | Lung cancer |
| Dermatology | Skin cancer | Skin cancer |
| Ear, Nose, | H&N cancer, parotid | H&N cancer, thyroid |
| and Throat | cancer | cancer |
| Gastrointestine | HCC, CRC, Gastric cancer, | HCC, CRC, Gastric |
| | Pancreatic cancer, | cancer, Pancreatic |
| | Esophageal cancer, | cancer, Esophageal |
| | Gallbladder cancer | cancer, Gallbladder |
| | | cancer |
| Genitourinary | Bladder cancer, RCC | Prostate cancer, |
| | | Bladder cancer, RCC |
| Hematology | Leukemia, Lymphoma | Leukemia, Lymphoma |
| Neurology | CNS cancer | CNS cancer |
| Gynecological | Cervical cancer, Ovarian | NA |
| | cancer, Uteral cancer | |

Metrics Used for Comparison of Various Models for Cancers Screening

The metrics, including sensitivity, specificity, accuracy, positive predictive value (PPV), ROC curve, and AUROC were used to access and compare the performance of the ML models.

Statistical Analyses

A Chi-squared test was used to analyze the distribution of cancer cases among training and independent testing datasets, and Fisher's exact test was used for analysis when case number was less than 5. The confidence intervals for sensitivity, specificity, and accuracy were estimated using the calculation of the confidence interval for a proportion in one sample situation. Furthermore, the confidence intervals of AUROCs were determined using the nonparametric approach. The AUROCs were compared by a nonparametric approach proposed by Delong et al. [23] and the data analysis plan (MAQC) proposed by Shi et al. [24] was followed. Both internal cross validation and external validation with independent testing data were used to test the robustness/reproducibility of the machine learning approach.

### Example 2: Generation of the Classifier Models

Algorithm development was performed using the datasets obtained from CGMH Linkou branch. The male dataset consisted of tumor biomarker values and age from 8415 subjects, while the female dataset consisted of tumor biomarker values and age from 10,211 subjects. To train the ML algorithms, a balanced subset of subjects ultimately diagnosed with cancer and subjects who were not diagnosed within the follow-up period was randomly selected. The biomarker values and ages of these subjects were used to train the algorithms. This subsampling process was performed 200 times (random subsampling cross-validation) and then internally validated against the entire dataset. Completely independent datasets for both males (n=4207) and females (n=5105) were obtained from CGMH Kaohsioung branch and used for independent validation. Mean and median biomarker values for the training and independent datasets are reported in Table 1A. The statistics in Table 1A are from one random subsampling. As expected, due to the selection of balanced training data vs. the real world nature of the independent dataset, mean biomarker values vary widely, while median values are much more consistent between the datasets. Thus, median with interquartile range (IQR) is a more appropriate metric to compare groups. Based on these values, the distribution of tumor marker values in training datasets overlapped with those in the independent testing datasets. The distribution of cancer types within the training and independent datasets for both men (Table 1B), and women (Table 1C) are comparable. There are 19 cancer types in both groups. In the overall datasets, the top three cancer types for men originated from the colon, liver, and prostate while for women, the top three were breast, cervical, and thyroid.

TABLE 1A*
Descriptive statistics of training and independent testing datasets

| Variation | Median | IQR | Mean | SE | Median | IQR | Mean | SE |
|---|---|---|---|---|---|---|---|---|
| Male Training (n = 248) | | | | | Male Independent Testing (n = 4207) | | | |
| AFP (ng/mL) | 3.40 | 1.99 | 2100.40 | 23,452.06 | 3.08 | 1.75 | 4.26 | 44.68 |
| CEA (ng/mL) | 2.01 | 1.68 | 3.62 | 10.56 | 1.84 | 1.44 | 2.20 | 1.52 |
| CA19-9 (U/mL) | 6.37 | 9.48 | 10.85 | 16.32 | 5.09 | 7.90 | 7.77 | 11.42 |
| CYFRA21-1 | 1.61 | 0.98 | 1.86 | 1.15 | 1.35 | 0.91 | 1.54 | 0.83 |
| (ng/mL) | | | | | | | | |
| SCC (ng/mL) | 0.40 | 0.50 | 0.61 | 0.51 | 0.50 | 0.50 | 0.64 | 0.64 |
| PSA (ng/mL) | 1.14 | 1.28 | 8.92 | 103.62 | 0.81 | 0.80 | 1.25 | 2.13 |
| Age (yr) | 57.00 | 20.25 | 57.43 | 13.42 | 49.00 | 18.00 | 49.55 | 12.65 |
| Female Training (n = 208) | | | | | Female Independent Testing (n = 5105) | | | |
| AFP (ng/mL) | 3.01 | 1.63 | 4.69 | 12.41 | 2.81 | 1.75 | 3.35 | 4.45 |
| CEA (ng/mL) | 1.42 | 1.18 | 3.83 | 21.21 | 1.27 | 1.02 | 1.52 | 1.07 |
| CA19-9 (U/mL) | 5.72 | 9.86 | 11.82 | 40.84 | 6.19 | 10.28 | 9.51 | 11.57 |
| CYFRA21-1 | 1.34 | 1.07 | 1.70 | 1.37 | 1.16 | 0.76 | 1.32 | 0.76 |
| (ng/mL) | | | | | | | | |
| SCC (ng/mL) | 0.30 | 0.30 | 0.54 | 0.84 | 0.30 | 0.30 | 0.49 | 0.40 |
| CA125 (U/mL) | 9.80 | 8.19 | 15.29 | 17.63 | 9.39 | 6.84 | 12.13 | 14.00 |
| CA153 (U/mL) | 8.70 | 5.78 | 10.30 | 5.21 | 8.20 | 5.60 | 9.50 | 4.38 |
| Age (yr) | 49.00 | 14.25 | 50.56 | 10.73 | 47.00 | 16.00 | 47.43 | 11.77 |

*IQR: interquartile range; SE: standard error

TABLE 1B
Cancer types in male training and independent testing datasets

| | Training Set | | | Independent | |
|---|---|---|---|---|---|
| Cancer Type | #Cancer | | #Cancer | Testing Set | |
| Male | (Total = 124) | (%) | (Total = 62) | (%) | p-Value |
| Prostate | 18 | 14.5 | 12 | 19.4 | 0.53 |
| Liver | 17 | 13.7 | 15 | 24.2 | 0.11 |
| Colon | 14 | 11.3 | 6 | 9.7 | 0.93 |
| Lung | 9 | 7.3 | 1 | 1.6 | 0.21 |
| Pancreatic | 9 | 7.3 | 7 | 11.3 | 0.52 |
| Head and neck | 8 | 6.5 | 0 | 0.0 | 0.1 |
| Thyroid | 7 | 5.7 | 0 | 0.0 | 0.13 |
| Kidney | 6 | 4.8 | 6 | 9.7 | 0.34 |
| Leukemia | 6 | 4.8 | 3 | 4.8 | 1 |
| Gastric | 6 | 4.8 | 1 | 1.6 | 0.5 |
| Bladder | 5 | 4.0 | 1 | 1.6 | 0.66 |
| Lymphoma | 5 | 4.0 | 3 | 4.8 | 1 |
| Skin | 5 | 4.0 | 2 | 3.2 | 1 |
| Esophageal | 2 | 1.6 | 3 | 4.8 | 0.42 |
| Unknown origin | 2 | 1.6 | 0 | 0.0 | 0.8 |
| Bile ducts | 2 | 1.6 | 0 | 0.0 | 1 |
| Brain | 1 | 0.8 | 1 | 1.6 | 1 |
| Retroperitoneal | 1 | 0.8 | 0 | 0.0 | 1 |
| Testicle | 1 | 0.8 | 0 | 0.0 | 1 |
| Gastrointestinal | 0 | 0.0 | 1 | 1.6 | 0.72 |
| stromal | | | | | |

TABLE 1C
Cancer types in female training and independent testing datasets

| | Training Set | | | Independent | |
|---|---|---|---|---|---|
| Cancer Type | #Cancer | | #Cancer | Testing Set | |
| Female | (Total = 104) | (%) | (Total = 52) | (%) | p-Value |
| Breast | 31 | 29.8 | 27 | 51.9 | 0.01 |
| Cervical | 17 | 16.4 | 2 | 3.9 | 0.05 |
| Thyroid | 15 | 14.4 | 5 | 9.6 | 0.55 |
| Colon | 9 | 8.7 | 2 | 3.9 | 0.44 |
| Lung | 5 | 4.8 | 0 | 0.0 | 0.26 |
| Liver | 4 | 3.9 | 3 | 5.8 | 0.89 |
| Ovarian | 4 | 3.9 | 1 | 1.9 | 0.87 |
| Gastric | 4 | 3.9 | 1 | 1.9 | 0.87 |
| Kidney | 2 | 1.9 | 2 | 3.9 | 0.86 |
| Leukemia | 2 | 1.9 | 1 | 1.9 | 1 |
| Skin | 2 | 1.9 | 2 | 3.9 | 0.86 |
| Unknown origin | 2 | 1.9 | 0 | 0.0 | 0.8 |
| Uterus | 2 | 1.9 | 3 | 5.8 | 0.42 |
| Bladder | 1 | 1.0 | 1 | 1.9 | 1 |
| Head and neck | 1 | 1.0 | 0 | 0.0 | 1 |
| Liposarcoma | 1 | 1.0 | 0 | 0.0 | 1 |
| Nasal | 1 | 1.0 | 0 | 0.0 | 1 |
| neuroendocrine | | | | | |
| tumor | | | | | |
| Pancreatic | 1 | 1.0 | 0 | 0.0 | 1 |
| Esophageal | 0 | 0.0 | 1 | 1.9 | 0.72 |
| Parotid | 0 | 0.0 | 1 | 1.9 | 0.72 |

Performance of the Classifier Models

For men, ML models using a LR algorithm consistently outperformed RF and SVM models in either internal (Table 2A and FIG. 1a) or external validation (Table 2A and FIG. 1b) in terms of AUROC. Specific cutoff values for risk scores can be applied to define low, mild, moderate, and high-risk groups (FIG. 7). The PPVs of ML models using LR algorithm were 1.99%, 2.89%, and 11.72% for mild, moderate, and high-risk groups, respectively. Test performance within the top three male cancers was comparable (Table 2B and FIG. 1c). For women, ML models using RF algorithm outperformed LR and SVM (Table 2A and FIG. 1d,e). Overall model performance characteristics for the female algorithms were inferior to the male algorithm, but still yielded superior performance to single marker tests (Table 2A) [3]. In the subgroup analysis, the ML models showed comparable performance in detecting the top three female cancers (Table 2B and FIG. 1f). As with the male models, specific cutoff values for risk scores can be applied to the female model to define low, mild, moderate, and high-risk groups (FIG. 7). The PPVs of ML models using the RF algorithm were 1.66%, 3.74%, and 10.87% for mild, moderate, and high-risk female groups, respectively. The PPVs were calculated based on the entire combined dataset (including both training datasets and testing datasets).

TABLE 2A**
Performance of cancers screening algorithms

| Male | LR | RF | SVM |
|---|---|---|---|
| AUROC | | | |
| Internal CV | 0.7654 (0.7596, 0.7713) | 0.7555 (0.7499, 0.7612) | 0.7440 (0.7380, 0.7500) |
| External validation | 0.8736 (0.8347, 0.9125) | 0.8382 (0.7984, 0.8781) | 0.6804 (0.5880, 0.7728) |
| MCC | | | |
| Internal CV | 0.0554 (0.0522, 0.0566) | 0.0551 (0.0529, 0.0573) | 0.0504 (0.0483, 0.0525) |
| External validation | 0.2092 (0.1969, 0.2214) | 0.1384 (0.1280, 0.1489) | 0.1217 (0.1119, 0.1316) |
| Weighted Accuracy | | | |
| Internal CV | 0.7076 (0.7032, 0.7120) | 0.7311 (0.7268, 0.7354) | 0.7201 (0.7157, 0.7244) |
| External validation | 0.8171 (0.8055, 0.8288) | 0.7686 (0.7558, 0.7813) | 0.7098 (0.6961, 0.7235) |
| Sensitivity | | | |
| Internal CV | 0.6604 (0.6597, 0.6611) | 0.6757 (0.6749, 0.6764) | 0.7028 (0.7021, 0.7035) |
| External validation | 0.7742 (0.7616, 0.7868) | 0.8387 (0.8276, 0.8498) | 0.6129 (0.5982, 0.6276) |
| Specificity | | | |
| Internal CV | 0.7418 (0.7412, 0.7425) | 0.7203 (0.7197, 0.7210) | 0.6996 (0.6989, 0.7003) |
| External validation | 0.8601 (0.8496, 0.8706) | 0.6984 (0.6846, 0.7123) | 0.8068 (0.7948, 0.8187) |

| Female | LR | RF | SVM |
|---|---|---|---|
| AUROC | | | |
| Internal CV | 0.6068 (0.5995, 0.6140) | 0.6665 (0.6596, 0.6733) | 0.5794 (0.5717, 0.5870) |
| External validation | 0.6181 (0.5428, 0.6935) | 0.6938 (0.6298, 0.7579) | 0.5551 (0.4834, 0.6268) |
| MCC | | | |
| Internal CV | 0.0218 (0.0205, 0.0231) | 0.0353 (0.0337, 0.0370) | 0.0272 (0.0258, 0.0287) |
| External validation | 0.0312 (0.0296, 0.0327) | 0.0562 (0.0542, 0.0582) | −0.0042 (−0.0048, −0.0036) |
| Weighted Accuracy | | | |
| Internal CV | 0.5818 (0.5774, 0.5861) | 0.6404 (0.6361, 0.6446) | 0.6052 (0.6009, 0.6095) |
| External validation | 0.5673 (0.5360, 0.5717) | 0.6349 (0.6307, 0.6392) | 0.4904 (0.4860, 0.4948) |
| Sensitivity | | | |
| Internal CV | 0.4850 (0.4843, 0.4857) | 0.5736 (0.5729, 0.5743) | 0.3298 (0.3292, 0.3305) |
| External validation | 0.5192 (0.5055, 0.5329) | 0.6923 (0.6796, 0.7050) | 0.2885 (0.2760, 0.3009) |
| Specificity | | | |
| Internal CV | 0.6657 (0.6651, 0.6664) | 0.6521 (0.6515, 0.6528) | 0.8007 (0.8002, 0.8013) |
| External validation | 0.6855 (0.6728, 0.6983) | 0.6139 (0.6005, 0.6272) | 0.6553 (0.6422, 0.6683) |

TABLE 2B**
Performance of screening top three types cancers

| Male | Colon Cancer | Liver Cancer | Prostate Cancer | Other |
|---|---|---|---|---|
| AUROC | 0.8290 (0.6660, 0.9921) | 0.8694 (0.7977, 0.9411) | 0.8608 (0.7961, 0.9255) | 0.9319 (0.9003, 0.9636) |
| Accuracy | 0.8106 (0.7987, 0.8224) | 0.8662 (0.8559, 0.8765) | 0.8448 (0.8338, 0.8557) | 0.8602 (0.8498, 0.8707) |
| Sensitivity | 0.8333 (0.8221, 0.8446) | 0.8000 (0.7879, 0.8121) | 1 | 0.7241 (0.7106, 0.7376) |
| Specificity | 0.8080 (0.7961, 0.8199) | 0.8649 (0.8546, 0.8752) | 0.8425 (0.8315, 0.8535) | 0.8601 (0.8496, 0.8706) |
| Female | Breast Cancer | Cervical Cancer | Thyroid Cancer | Other |
| AUROC | 0.6776 (0.5825, 0.7727) | 0.6235 (0.1628, 1.000) | 0.6940 (0.4286, 0.9594) | 0.7259 (0.6363, 0.8155) |
| Accuracy | 0.6453 (0.6322, 0.6584) | 0.6948 (0.6821, 0.7074) | 0.7444 (0.7324, 0.7563) | 0.5821 (0.5686, 0.5957) |
| Sensitivity | 0.5926 (0.5791, 0.6061) | 0.5000 (0.4863, 0.5137) | 0.6000 (0.5866, 0.6134) | 0.7778 (0.7664, 0.7862) |
| Specificity | 0.6456 (0.6324, 0.6587) | 0.6948 (0.6822, 0.7075) | 0.7445 (0.7325, 0.7565) | 0.58145679, 0.5950) |

**LR: logistic regression; RF: random forest; SVM: support vector machine; AUROC: area under the receiver operating characteristic curve; CV: cross-validation; MCC: Matthews correlation coefficient. The metrics are described with mean and 95% confidence interval.
AUROC: area under the receiver operating characteristic curve; Other: other cancer types. The metrics are described with mean and 95% confidence interval.

### Example 3: Individuals for Whom Earlier Retesting May have Detected their Cancers Earlier

The following are actual real-world examples of individuals who were diagnosed with cancer within 12 months of their last blood test who would have benefited from being retested at earlier time intervals based on methods according to the present invention.

In particular, these individuals all had tumor marker levels below standard “cut-offs” (i.e., the level above which an expert flags for follow-up) so they would not likely have received further testing absent signs or symptoms of disease. On the other hand, using the computer systems and methods according to the present invention they would have been recommend earlier retesting.

| | | | Tumor Markers | | |
|---|---|---|---|---|---|
| | | | Tested & Found | | |
| | | | to be “Normal” | Date of | Time to Retest |
| | | | (i.e., below the | Cancer | (Suggested by |
| Gender | Birthdate | Test Date | cutoff) | Diagnosis | Software) |
| Male | Sep. 15, 1931 | Apr. 9, 2010 | Normal | Dec. 16, 2010 | Visit specialist |
| | | | | | in 2 months |
| Male | Jul. 23, 1938 | May 4, 2010 | Normal | Aug. 5, 2010 | Visit specialist |
| | | | | | in 2 moths |
| Female | May 15, 1963 | May 7, 2012 | Normal | Dec. 11, 2012 | Retest in 1 month |
| Female | Jul. 13, 1957 | Aug. 18, 2009 | Normal | Jun. 15, 2010 | Visit specialist |
| | | | | | in 6 months |

### Example 4: Association Between the Risk Score, Cancer Stage, and Time-to-Diagnosis; Classifier Model for Time to Retest

Risk scores were positively correlated to the cancer stage (FIG. 3). For both men and women, disease stage 0, I, and II accounted for more than half of the cases whose risk was low, mild, or moderate. By contrast, in the high-risk group, the percentage of stage I cancers decreased and the percentage of stage III increased (FIG. 3). Regarding the time-to-diagnosis (TTD), the time interval between tumor marker test and cancer diagnosis was measured. For both men and women, the risk score was negatively correlated to the TTD (FIG. 4). For men, the median TTD was 561, 451.5, 204.5, and 28 days for low, mild, moderate, and high-risk groups, respectively (FIG. 4A). For women, the median TTD was 279, 229, 132, and 125 days for the low, mild, moderate, and high-risk groups, respectively (FIG. 4B).

Provided herein are generated and validated robust ML models capable of improving early detection of cancer and localizing the tissue origin by using RWD of tumor markers. These ML-derived algorithms are appropriate cancer screening tools demonstrating high levels of accuracy, generalizability, and affordability.

### Example 5: Association Between the Risk Score, Cancer Stage, and Time-to-Diagnosis (TTD); Classifier Model for Time to Retest

This example describes the use of LSTM architecture to develop a predictive algorithm to classify cancer risk from TM data. The algorithm takes as input single time-point values of one, two, three, or four biomarkers from the panel CEA, AFP, PSA and CA19-9 and uses a LSTM model to establish a second classifier model for predicting cancer likelihood. This model is developed using a larger dataset of real-world data and also is correlated to time-to-diagnosis.

Data was abstracted from health check-up databases was obtained from the First Affiliated Hospital of Chongqing Medical University (CHQ) and Chang Gung Memorial Hospital (CGMH) including subjects seen between May 2001 and December 2019. All subjects had one or more cancer biomarkers measured for screening purposes at the Department of Laboratory Medicine and did not report any symptoms associated with any cancer type at the time of testing. Subjects were followed up subsequent to the health check-up examination and medical records indicating disease status were observed for more than one year to determine the status of cancer diagnosis. Exclusion criteria included loss of follow-up, no further medical examination within one year, and cancer diagnosis before the analytical measurements of TM. This study was approved by the CHQ Ethics Committee (Approval Number: 2020-089). The study included >130,000 subjects as CHQ and >27,000 subjects at CGMH.

At the time of the health check-up examination, blood samples were drawn by venipuncture from all study subjects for the analytical measurement of one or more of the following TMs: AFP, CA15-3, CA125, PSA, SCC, CEA, CYFR21-1 and CA19-9. All TM levels were determined by an automated chemiluminescence immunoassay analyzer (Cobas 8000 e602, Roche Diagnostics Inc), except that SCC was measured using commercially available kits from Abbott Diagnostics, Abbott Park, Ill., USA). Clinical reference values for each of the tumor markers were as follows: 25 ng/ml for AFP, 25 U/ml for CA 15-3, 35 U/ml for CA125, 4 ng/ml for PSA, 1.5 ng/ml for SCC, 10 ng/ml for CEA, 3.3 ng/ml for CYFRA 21-1 and 27 U/ml for CA 19-9. All inspection processes have passed ISO15189 certification (NO. ML00036).

Follow-Up Criteria in CHQ

At CHQ, follow-up to a health check-up including the measure of TMs includes an analysis of the TM results combined with other relevant findings from the examination. The TM results of the subjects were classified as elevated if measured at twice the reference value and were grouped according to the results of other relevant examination items. It should be noted that this follow-up protocol was somewhat more aggressive than the follow-up protocols used at other institutions, including at CGMH.

Training, Internal Validation, and Validations of the LSTM Models

Repeated cross-validations were used to develop and validate the models. The variables included gender, the tumor marker values and age. Since the sourced data is real world data, the datasets were extremely imbalanced, with the ratio of cancer cases to non-cancer cases around 1:100 for CGMH data and 1:333 for Chongqing data. When using an extremely imbalanced dataset, there is a significant probability that a bootstrap sample contains few or even none of the minority class, resulting in a tree with poor performance for predicting the minority class¹⁰.

Time-to-Diagnosis Analysis

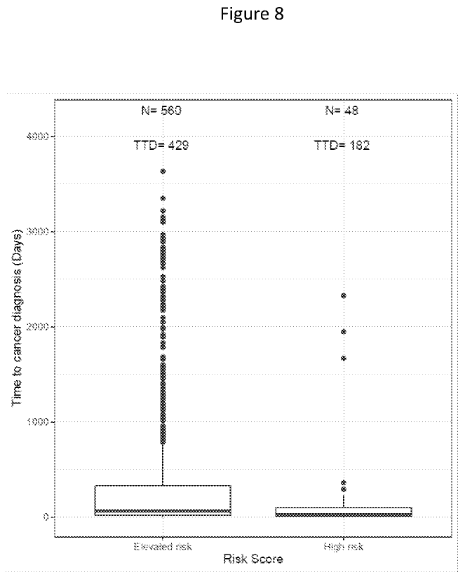

Time-to-event data analysis is widely used in oncology, such as the time from cancer diagnosis or treatment initiation to cancer recurrence or death. In this case a single cutoff was applied to the probability/likelihood scores generated by the LSTM, splitting the subjects into two groups, those with elevated risk and those with high risk. The time-to-diagnosis of individuals ultimately diagnosed with cancer in the two cohorts were analyzed and compared (FIG. 8). As indicated in the box plot, the median TTD for the elevated risk group was 429 days, while the median TTD for the high risk group was 182 days.

This example demonstrates that the time-to-diagnosis tool is relevant for multiple cohorts of real-world subjects and when using different ML-derived classifier models.

### Example 6: Application of the TTD Models

As shown above, the ML-derived first classifier models provide flexibility in working with complicated tumor marker tests in real-world and can be readily deployed in routine health check-ups. In one embodiment, the results provided by the machine learning-derived algorithm scores are converted to a positive predictive value PPV which is essentially a risk ratio (“OneTest′ Score”). The vast majority (>95%) of OneTest™ Scores, even most elevated scores, will be in the single digits. The highest possible OneTest™ Scores are −30 and even at this score only one in three individuals have cancer. Individuals with higher OneTest™ Scores are encouraged to follow up with a health care provider. The OneTest™ Score is one factor to be considered when determining whether additional and/or more frequent follow-up testing is warranted. Other factors can include, for instance, cancer signs and/or symptoms, abnormally high biomarker (BM, TM) levels, and/or rising BM and/or TM levels after two or more tests. Thus, OneTest™ is a screening test for cancer, and is not a diagnostic test. In general, the most appropriate follow-up to a suspicious result on a screening test is further testing, moving towards more definitive diagnostic tests.

Less than a 3% of individuals with a Low Risk OneTest™ Score (1-2) have cancer and those in this group who will develop cancer have a time to diagnosis (TTD) of >1 year. According to the US Cancer Data and Statistics/CDC, cancer incidence rates increase with age and range from 0.60%-2.7% from ages 50-85+ years old. That said, a OneTest™ Score of 1-2 does not suggest a higher risk of having cancer than in the general population. Furthermore, even cancer cases in our real-world cohort that had scores in this range were generally diagnosed more than two years out from the date of screening. As such the most likely follow-up for scores of 1 or 2 is simply to continue annual screening for cancer using OneTest™.

Three to four percent of individuals with a Mild Risk OneTest™ Score (3-4) have cancer, and those in this group who will develop cancer have a time to diagnosis (TTD) of >1 year. OneTest™ Scores in this range represent a two-fold increase in risk over the general population. While this is still a relatively low overall risk it may suggest heightened vigilance and what is often referred to as “watchful waiting”. In evaluating scores in this range, it may be helpful to also consider the individual biomarker results and whether any of these values are outside of the standard reference ranges. Cancer cases with scores in this range in our real-world cohort were generally diagnosed more than one year out from the date of screening. The suggested follow-up for patients with scores of three or four might include greater frequency of repeat testing with OneTest™, in some embodiments approximately every six months. In cases where a biomarker is also elevated above the reference range or there is some other concerning factor (e.g., family history of cancer), a health care provider may suggest more definitive testing.

Five to eight percent of individuals with a Moderate Risk OneTest Score (5-8) have cancer, and those in this group who will develop cancer have a time to diagnosis (TTD) of six to 12 months. Scores in this range suggest that one out of every 14-20 individuals will have cancer. The median time to diagnosis for cancer cases identified in our real-world cohort with scores in this range was less than one year. Individuals with scores in this range should therefore be followed much more closely. Possible follow-up may include more definitive diagnostic tests or increased frequency of screening with OneTest′ within approximately three to four months. The health care provider will likely consider other factors including elevated single markers and/or other clinical factors like family history of cancer in determining the most appropriate follow-up.

Nine percent of individuals with a High Risk OneTest™ Score (>9) have cancer, and those in this group who will develop cancer have a time to diagnosis (TTD) of two to four months. Testing of an exemplary cohort showed that 50% of all active cancer cases had scores in this range. Furthermore, only 10% of individuals not found to have cancer in the follow up period had scores in this range. More than 1 out of every 13 individuals in this group will be diagnosed with cancer. Median time to diagnosis in this group was less than 6 months. As such, individuals with scores of nine and up should receive extensive follow-up testing as soon as possible under the advice of a health care provider.

While certain embodiments have been described in terms of the preferred embodiments, it is understood that variations and modifications will occur to those skilled in the art. Therefore, it is intended that the appended claims cover all such equivalent variations that come within the scope of the following claims.

### REFERENCES

1. Siegel, R. L.; Miller, K. D.; Jemal, A. Cancer Statistics, 2017. CA Cancer J. Clin. 2017, 67, 7-30.
2. Vogelstein, B.; Kinzler, K. W. The Path to Cancer—Three Strikes and You're Out. N Engl. J. Med. 2015, 373, 1895-1898.
3. Wang, H.-Y.; Hsieh, C.-H.; Wen, C.-N.; Wen, Y.-H.; Chen, C.-H.; Lu, J.-J. Cancers Screening in an Asymptomatic Population by Using Multiple Tumour Markers. PLoS ONE 2016, 11, e0158285.
4. Goncalves, A. R.; Ferreira, C.; Marques, A.; Ribeiro, L. C.; Velosa, J. Assessment of quality in screening colonoscopy for colorectal cancer. Clin. Exp. Gastroenterol. 2011, 4, 277-281.
5. Evans, A.; Trimboli, R. M.; Athanasiou, A.; Balleyguier, C.; Baltzer, P. A.; Bick, U.; Herrero, J. C.; Clauser, P.; Colin, C.; Cornford, E.; et al. Breast ultrasound: Recommendations for information to women and referring physicians by the European Society of Breast Imaging. Insights Imaging 2018, 9, 449-461.
6. Smith, R. A.; Andrews, K. S.; Brooks, D.; Fedewa, S. A.; Manassaram-Baptiste, D.; Saslow, D.; Brawley, 0.W.; Wender, R. C. Geographic Availability of Low-Dose Computed Tomography for Lung Cancer Screening in the United States, 2017. Prev. Chronic. Dis. 2018, 15, 119.
7. Koscielniak-Merak, B.; Radosavljevic, B.; Zajac, A.; Tomasik, P. J. Faecal Occult Blood Point-of-Care Tests. J. Gastrointest. Cancer 2018, 49, 402-405.
8. Huguet, N.; Angier, H.; Rdesinski, R.; Hoopes, M.; Marino, M.; Holderness, H.; DeVoe, J. E. Cervical and colorectal cancer screening prevalence before and after Affordable Care Act Medicaid expansion. Prev. Med. 2019, 124, 91-97.
9. Wen, Y. H.; Chang, P. Y.; Hsu, C. M.; Wang, H. Y.; Chiu, C. T.; Lu, J. J. Cancer screening through a multi-analyte serum biomarker panel during health check-up examinations: Results from a 12-year experience. Clin. Chim. Acta 2015, 450, 273-276.
10. Cohen, J. D.; Li, L.; Wang, Y.; Thoburn, C.; Afsari, B.; Danilova, L.; Douville, C.; Javed, A. A.; Wong, F.; Mattox, A.; et al. Detection and localization of surgically resectable cancers with a multi-analyte blood test. Science 2018, 359, 926-930.
11. Palmirotta, R.; Lovero, D.; Cafforio, P.; Felici, C.; Mannavola, F.; Pellè, E.; Quaresmini, D.; Tucci, M.; Silvestris, F. Liquid biopsy of cancer: A multimodal diagnostic tool in clinical oncology. Ther. Adv. Med. Oncol. 2018, 10, 1758835918794630.
12. Aravanis, A. M.; Lee, M.; Klausner, R. D. Next-Generation Sequencing of Circulating Tumor DNA for Early Cancer Detection. Cell 2017, 168, 571-574.
13. Sherman, R. E.; Anderson, S. A.; Dal Pan, G. J.; Gray, G. W.; Gross, T.; Hunter, N. L.; LaVange, L.; Marinac-Dabic, D.; Marks, P. W.; Robb, M. A.; et al. Real-World Evidence—What Is It and What Can It Tell Us? N Engl. J. Med. 2016, 375, 2293-2297.
14. Corrigan-Curay, J.; Sacks, L.; Woodcock, J. Real-World Evidence and Real-World Data for Evaluating Drug Safety and Effectiveness. JAMA 2018, 320, 867-868.
15. Marino, P.; Touzani, R.; Perrier, L.; Rouleau, E.; Kossi, D. S.; Zhaomin, Z.; Charrier, N.; Goardon, N.; Preudhomme, C.; Durand-Zaleski, I.; et al. Cost of cancer diagnosis using next-generation sequencing targeted gene panels in routine practice: A nationwide French study. Eur. J. Hum. Genet. 2018, 26, 314-323.
16. Lin, W. Y.; Chen, C. H.; Tseng, Y. J.; Tsai, Y. T.; Chang, C. Y.; Wang, H. Y.; Chen, C. K. Predicting post-stroke activities of daily living through a machine learning-based approach on initiating rehabilitation. Int. J. Med. Inform. 2018, 111, 159-164.
17. Wang, H. Y.; Lee, T. Y.; Tseng, Y. J.; Liu, T. P.; Huang, K. Y.; Chang, Y. T.; Chen, C. H.; Lu, J. J. A new scheme for strain typing of methicillin-resistant Staphylococcus aureus on the basis of matrix-assisted laser desorption ionization time-of-flight mass spectrometry by using machine learning approach. PLoS ONE 2018, 13, e0194289.
18. Wang, H. Y.; Lee, T. Y.; Tseng, Y. J.; Liu, T. P.; Huang, K. Y.; Chang, Y. T.; Chen, C. H.; Lu, J. J. Rapid Detection of Heterogeneous Vancomycin-Intermediate Staphylococcus aureus Based on Matrix-Assisted Laser Desorption Ionization Time-of-Flight: Using a Machine Learning Approach and Unbiased Validation. Front. Microbiol. 2018, 9, 2393.
19. Bossuyt, P. M.; Reitsma, J. B.; Bruns, D. E.; Gatsonis, C. A.; Glasziou, P. P.; Irwig, L.; Lijmer, J. G.; Moher, D.; Rennie, D.; De Vet, H. C.; et al. STARD 2015: An updated list of essential items for reporting diagnostic accuracy studies. BMJ 2015, 351, h5527.
20. Núñez, H.; Gonzalez-Abril, L.; Angulo, C. Improving SVM Classification on Imbalanced Datasets by Introducing a New Bias. J. Classif 2017, 34, 427-443.
21. Breiman, L. Random Forest. Mach. Learn. 2001, 45, 5-32.
22. Chen, C.; Liaw, A.; Breiman, L. Using random forest to learn imbalanced data. Univ. Calif. Berkeley 2004, 110, 24.
23. DeLong, E. R.; DeLong, D. M.; Clarke-Pearson, D. L. Comparing the Areas under Two or More Correlated Receiver Operating Characteristic Curves: A Nonparametric Approach. Biometrics 1988, 44, 837-845.
24. Shi, L.; Campbell, G.; Jones, W.; Campagne, F.; Wen, Z.; Walker, S.; Su, Z.; Chu, T.; Goodsaid, F.; Pusztai, L.; et al. The MAQC-II Project: A Comprehensive Study of Common Practices for the Development and Validation of Microarray-Based Predictive Models. 2010. Available online: https://cris.fbk.eu/handle/11582/10568#.XtBlyPwRVPY (accessed on 15 May 2020).
25. Pinsky, P. F.; Prorok, P. C.; Kramer, B. S. Prostate Cancer Screening—A Perspective on the Current State of the Evidence. N. Engl. J. Med. 2017, 376, 1285-1289.
26. Molina, R.; Marrades, R. M.; Augé, J. M.; Escudero, J. M.; Viñolas, N.; Reguart, N.; Ramirez, J.; Filella, X.; Molins, L.; Agusti, A. Assessment of a Combined Panel of Six Serum Tumor Markers for Lung Cancer. Am. J. Respir. Crit. Care Med. 2016, 193, 427-437.
27. Doseeva, V.; Colpitts, T.; Gao, G.; Woodcock, J.; Knezevic, V. Performance of a multiplexed dual analyte immunoassay for the early detection of non-small cell lung cancer. J. Transl. Med. 2015, 13,55.
28. Shah, N. H.; Milstein, A.; Bagley Ph, D. S. Making Machine Learning Models Clinically Useful. JAMA 2019, 322, 1351-1352.
29. Shim, J. H.; Han, S.; Lee, Y. J.; Lee, S. G.; Kim, K. M.; Lim, Y. S.; Chung, Y. H.; Lee, Y. S.; Lee, H. C. Half-life of serum alpha-fetoprotein: An early prognostic index of recurrence and survival after hepatic resection for hepatocellular carcinoma. Ann. Surg. 2013, 257, 708-717.
30. Chung, C. R.; Wang, H. Y.; Lien, F.; Tseng, Y. J.; Chen, C. H.; Lee, T. Y.; Horng, J. T.; Lu, J. J. Incorporating Statistical Test and Machine Intelligence Into Strain Typing of Staphylococcus haemolyticus Based on Matrix-Assisted Laser Desorption Ionization-Time of Flight Mass Spectrometry. Front. Microbiol. 2019, 10, 2120.
31. Tseng, Y. J.; Huang, C. E.; Wen, C. N.; Lai, P. Y.; Wu, M. H.; Sun, Y. C.; Wang, H. Y.; Lu, J. J. Predicting breast cancer metastasis by using serum biomarkers and clinicopathological data with machine learning technologies. Int. J. Med. Inform. 2019, 128, 79-86.
32. Wang, H. Y.; Lu, K. P.; Chung, C. R.; Tseng, Y. J.; Lee, T. Y.; Chang, T. H.; Wu, M. H.; Lin, T. W.; Liu, T. P.; Lu, J. J. Rapidly predicting vancomycin resistance of Enterococcus faecium through MALDI-TOF MS spectrum obtained in real-world clinical microbiology laboratory. bioRxiv 2020, doi:10.1101/2020.03.13.990978.
33. Wang, H. Y.; Hung, C. C.; Chen, C. H.; Lee, T. Y.; Huang, K. Y.; Ning, H. C.; Lai, N. C.; Tsai, M. H.; Lu, L. C.; Tseng, Y. J.; et al. Increase Trichomonas vaginalis detection based on urine routine analysis through a machine learning approach. Sci Rep. 2019, 9, 11074.
34. Wattal, C.; Oberoi, J. K.; Goel, N.; Raveendran, R.; Khanna, S Rapid classification of group B Streptococcus serotypes based on matrix-assisted laser desorption ionization-time of flight mass spectrometry and machine learning techniques. BMC Bioinform. 2019, 20 (Suppl. S19), 703.
35. Preissner, C. M.; Dodge, L. A.; O'Kane, D. J.; Singh, R. J.; Grebe, S. K. Prevalence of heterophilic antibody interference in eight automated tumor marker immunoassays. Clin. Chem. 2005, 51, 208-210.
36. Esteghamati, A.; Hafezi-Nejad, N.; Zandieh, A.; Sheikhbahaei, S.; Emamzadeh-Fard, S.; Nakhjavani, M. CA 19-9 is associated with poor glycemic control in diabetic patients: Role of insulin resistance. Clin. Lab. 2014, 60, 441-447.
37. Samarasinghe, S.; Meah, F.; Singh, V.; Basit, A.; Emanuele, N.; Emanuele, M. A.; Mazhari, A.; Holmes, E. W. Biotin Interference with Routine Clinical Immunoassays: Understand the Causes and Mitigate the Risks. Endocr. Pract. 2017, 23, 989-998.
38. Islami, F.; Miller, K. D.; Siegel, R. L.; Zheng, Z.; Zhao, J.; Han, X.; Ma, J.; Jemal, A.; Yabroff, K. R. National and State Estimates of Lost Earnings From Cancer Deaths in the United States. JAMA Oncol. 2019, 5, e191460.
39. Oh, J.; Makar, M.; Fusco, C.; McCaffrey, R.; Rao, K.; Ryan, E. E.; Washer, L.; West, L. R.; Young, V. B.; Guttag, J.; et al. A generalizable, data-Driven approach to predict daily risk of Clostridium difficile infection at two large academic health centers. Infect. Control Hosp. Epidemiol. 2018, 39, 425-433.

What is claimed is:

1. A method for identifying a patient for follow-up cancer diagnostic testing, the method comprising:
a) assigning a risk score of having or developing cancer to the patient, wherein the risk score is generated using a first classifier model using input variables of measured values of a panel of biomarkers from the patient and clinical factors including at least age and a diagnostic indicator, for a population of patients, when an output of the first classifier model is a numerical expression of the percent likelihood of having or developing cancer;
b) classifying the patient into an increased risk category of having or developing cancer when their risk score, generated by the first classifier model, is above a first pre-determined threshold, wherein the first pre-determined threshold is a prevalence of cancer in the population of patients;
c) classifying those patients in the increased risk category into a follow-up category using the risk score generated by the first classifier model, wherein a second pre-determined threshold, and optionally third pre-determined threshold, separate the follow-up categories and the second, and optionally third, pre-determined threshold is a median time to definitive diagnosis following measurement of the biomarkers of the population of patients; and,
d) providing a notification to a user of the patient risk score and follow-up category, wherein follow-up testing is selected from repeat testing in about one year, repeat testing in less than about 1 year and/or confirmatory cancer diagnostic testing.

2. A computer-implemented method for generating a follow-up cancer diagnostic testing classifier model comprising:
a) obtaining, by one or more processors, a data set from a population of patients comprising a risk score of having or developing cancer and time to definitive diagnosis following measurement of one or more biomarkers, wherein the risk score is generated by a first classifier model using inputs of measured values of the one or more biomarkers, optionally age, and a diagnostic indicator, from a population of patients;
b) segmenting the data into two or more groups based on the risk score; and,
c) determining a median time to definitive diagnosis in each group; and,
d) generating the classifier model for follow-up cancer diagnostic testing based on the correlation between the risk score and the time to definitive diagnosis, wherein the classifier model provides output selected from repeat testing in about one year, repeat testing in less than about 1 year and/or confirmatory cancer diagnostic testing.

3. A method for screening for cancers in an asymptomatic human subject comprising:
a. obtaining a first blood sample from the human subject;
b. measuring a panel of at least two markers in the sample, wherein said markers are selected from the group consisting of CEA, AFP, CA125, CA15-3, CA19-9, Cyfra, and PSA;
c. providing trained machine learning software to produce a cancer likelihood score, wherein said software is trained using data from individuals previously tested with said marker panel and for which cancer outcomes are known;
d. generating a cancer likelihood score for the human subject;
e. using said cancer likelihood score to calculate the optimal time interval when said at least two markers should be re-measured in a second blood sample from the human subject;
f. obtaining a second blood sample from the human subject based on said time interval; and
g. re-measuring said panel of markers in the second blood sample and comparing the changes in marker levels between the first and second blood samples.
